(19) United States
(12) Patent Application Publication
(10) Pub. No.: US 2004/0101956 A1
(43) Pub. Date: May 27, 2004

(54) Novel g protein-coupled receptor protein

(76) Inventors: Yasuko Terao, Kobe-shi (JP); Yasushi Shintani, Toyonaka-shi (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/362,504

(22) PCT Filed: Aug. 23, 2001
(86) PCT No.: PCT/JP01/07209

(30) Foreign Application Priority Data
Aug. 24, 2000 (JP) 2000-253862

(51) Int. Cl. C12N 15/11; C07K 14/705; C12P 21/02; A61K 38/17; G01N 33/53

(52) U.S. Cl. 435/320.1; 530/350; 435/69.1; 435/325; 435/7.1; 514/12

(57) ABSTRACT

The present invention intends to provide a novel protein, which is useful in a screening of agonist/antagonist, etc.

Specifically, the present invention provides a rat-derived protein or a salt thereof, a DNA encoding the same, a method for determination of ligand to the protein, a screening method/kits for a compound or its salt that alters a binding property between ligand and the protein, a compound or its salt obtained by the screening, and the like.

### TECHNICAL FIELD

The present invention relates to a novel protein (G protein-coupled receptor protein) derived from human brain or its salts and DNA encoding the same, a method for determination of ligand to the same, a compound that alters a binding property to the ligand, or a salt thereof, etc.

### BACKGROUND ART

Various hormones and neurotransmitters regulate the biological function via specific receptor proteins present on cell membranes. Many of these receptor proteins are coupled with guanine nucleotide-binding protein (hereinafter sometimes simply referred to as G protein) and mediate the intracellular signal transduction via activation of G protein. These receptor proteins possess the common structure containing seven transmembrane domains and are thus collectively referred to as G protein-coupled receptors or seven-transmembrane receptors.

G protein-coupled receptor proteins present on the cell surface of each functional cell and organ in the body, and play important physiological roles as the target of the molecules that regulate the functions of the cells and organs, e.g., hormones, neurotransmitters, physiologically active substances and the like.

To clarify the relationship between substances that regulate complex biological functions in various cells and organs, and their specific receptor proteins, in particular, G protein-coupled receptor proteins, would elucidate the functional mechanisms in various cells and organs in the body to provide a very important means for development of drugs closely associated with the functions.

For example, in organs of central nerve system such as brain, physiological functions in brain are controlled in vivo through regulation by many hormones, hormone-like substances, neurotransmitters or physiologically active substances. In particular, physiologically active substances are found in numerous sites of the body and regulate the physiological functions through their corresponding receptor proteins. However, it is supposed that many unknown neurotransmitters still exist in the brain and, as to structure of cDNA encoding the receptor proteins, it is considered many of those have not yet been reported. In addition, it is still unknown if there are subtypes of known receptor proteins.

It is very important for development of drugs to clarify the relationship between substances that regulate elaborated functions in brain and their specific receptor proteins. Furthermore, for efficient screening of agonists and antagonists to receptor proteins in development of drugs, it is required to clarify functional mechanisms of receptor protein genes expressed in brain and express the genes in an appropriate expression system.

In recent years, random analysis of cDNA sequences has been actively studied as a means for analyzing genes expressed in vivo. The sequences of cDNA fragments thus obtained have been registered on and published to databases as Expressed Sequence Tag (EST). However, since many ESTs contain sequence information only, it is difficult to predict their functions from the information.

The present invention provides a novel protein (G protein-coupled receptor protein) derived from rat brain, its partial peptides or salts thereof, DNA containing the DNA encoding the protein or its partial peptides, recombinant vectors containing the DNA, transformants transformed by the recombinant vectors, methods for manufacturing the protein or its salts, antibodies to the protein, its partial peptides or salts thereof, methods for determination of ligands to the protein (the G protein-coupled receptor protein), methods for screening compounds or salts thereof that alter the binding property between ligands and the protein (the G protein-coupled receptor protein), kits for use in the screening methods, compounds or salts thereof that alter the binding property between ligands and the protein (the G protein-coupled receptor protein) which is obtainable by the screening methods or the screening kits, and pharmaceuticals comprising the compounds or salts thereof that alter the binding property between ligands and the protein (the G protein-coupled receptor protein).

### DISCLOSURE OF THE INVENTION

The present inventors isolated cDNA encoding ZAQ, the G protein-coupled receptor protein derived from human brain. In addition, they have found that the protein ZAQ binds to Mamba Intestinal Toxin 1 (sometimes referred to as MIT1; Toxicon 28, 847-856 (1990); FEBS Letters 461, 183-188 (1999)) or its homologues derived from mammals (WO 01/16309).

As a result of extensive investigations, the present inventors have succeeded in isolating cDNAs encoding novel proteins (G protein-coupled receptor proteins derived from rat brain), and in sequencing the full-length base sequences. When the base sequences were translated into the amino acid sequences, 1 to 7 transmembrane domains were found to be on the hydrophobic plot, establishing that the proteins encoded by these cDNAs are seven-transmembrane type G protein-coupled receptor proteins (FIGS. 7 and 8). The present inventors have continued further extensive studies and as a result, have come to accomplish the present invention.

Thus, the present invention provides the following features.

(1) A G protein-coupled receptor protein containing the same or substantially the same amino acid sequence as the amino acid sequence represented by SEQ ID NO: 4 or SEQ ID NO: 11, or a salt thereof.

(2) A G protein-coupled receptor protein according to (1) which contains an amino acid sequence represented by SEQ ID NO: 4, or a salt thereof.

(3) A G protein-coupled receptor protein according to (1) which contains an amino acid sequence represented by SEQ ID NO: 11, or a salt thereof.

(4) A partial peptide of the protein according to (1), or a salt thereof.

(5) A polynucleotide containing a polynucleotide encoding the protein according to (1).

(6) A polynucleotide according to (5), which is DNA.

(7) The DNA according to (6), which contains a base sequence represented by SEQ ID NO: 3 or SEQ ID NO: 10.

(8) A recombinant vector containing the polynucleotide according to (5).

(9) A transformant transformed with the recombinant vector according to (8).

(10) A method of manufacturing the protein or its salt according to (1), which comprises culturing the transformant according to (9), producing and accumulating the protein according to (1).

(11) An antibody to the protein according to (1), the partial peptide according to (4), or a salt thereof.

(12) A method of determining a ligand to the protein or its salt according to (1), which comprises using the protein according to (1), the partial peptide according to (4), or a salt thereof.

(13) A method of screening a compound or its salt that alters the binding property between a ligand and the protein or its salt according to (1), which comprises using the protein according to (1), the partial peptide according to (4), or a salt thereof.

(14) A kit of screening a compound or its salt that alters the binding property between a ligand and the protein or its salt according to (1), which comprises using the protein according to 41), the partial peptide according to (4), or a salt thereof.

(15) A compound or its salt that alters the binding property between a ligand and the protein or its salt according to (1), which is obtainable using the screening method according to (13) or the screening kit according to (14).

(16) A pharmaceutical comprising a compound or its salt that alters the binding property between a ligand and the protein or its salt according to (1), which is obtainable using the screening method according to (13) or the screening kit according to (14).

(17) The pharmaceutical according to (16), which is a prophylactic or therapeutic agent for alimentary diseases.

(18) A DNA that hybridizes to the DNA according to (6) under a highly stringent condition.

(19) A prophylactic or therapeutic method for alimentary diseases, which comprises administering an effective amount of compound that alters a binding property between ligand and the protein according to (1) or its salt obtainable by using the screening method according to (13) or the screening kit according to (14), to mammals.

(20) Use of the compound that alters a binding property between ligand and the protein according to (1) or its salt obtainable by using the screening method according to (13) or the screening kit according to (14) for manufacturing a prophylactic or therapeutic agent for alimentary diseases.

More specifically, the present invention provides:

(21) A protein or its salt according to (1), wherein said protein contains \mathring{1} the amino acid sequence shown by SEQ ID NO: 4 or SEQ ID NO: 11, of which at least 1 or 2 (preferably approximately 1 to 30, more preferably approximately 1 to 9, most preferably several (1 to 2)) amino acids are deleted, \mathring{2} the amino acid sequence shown by SEQ ID NO: 4 or SEQ ID NO: 11, to which at least 1 or 2 (preferably approximately 1 to 30, more preferably approximately 1 to 10, most preferably several (1 to 2)) amino acids are added; \mathring{3} the amino acid sequence shown by SEQ ID NO: 4 or SEQ ID NO: 11, in which at least 1 or 2 (preferably approximately 1 to 30, more preferably approximately 1 to 10, most preferably several (1 to 2)) amino acids are substituted to other amino acids; or \mathring{4} the amino acid sequence containing a combination of these amino acid sequences;

(22) A method of determining a ligand according to (12), which comprises contacting the G protein-coupled receptor protein or its salt according to (1) or the partial peptide or its salt according to (4) with a test compound;

(23) A method of determining a ligand according to (12), in which said ligand is angiotensin, bombesin, canavinoid, cholecystokinin, glutamine, serotonin, melatonin, neuropeptide Y, an opioid, a purine, vasopressin, oxytocin, PACAP, secretin, glucagon, calcitnonin, adrenomedulin, somatostatin, GHRH, CRF, ACTH, GRP, PTH, vasoactive intestinal and related polypeptide (VIP), somatostatin, dopamine, motilin, amylin, bradykinin, calcitonin gene-related peptide (CGRP), a leukotriene, pancreastatin, a prostaglandin, thromboxane, adenosine, adrenaline, α- and β-chemokines (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide, galanin, MIT1 or their homologues derived from the mammal;

(24) A method of screening according to (13), in which (i) contact of a ligand with the protein or its salt according to (1) or the partial peptide or its salt according to (4) is compared with (ii) contact of the ligand and a test compound with the protein or its salt according to (1) or the partial peptide or its salt according to (4);

(25) A method of screening a compound or its salt that alters the binding property between a ligand and the protein or its salt according to (1), which comprises measuring the amounts of a labeled ligand bound to the protein or its salt according to (1) or to the partial peptide or its salt according to (4), (i) when the labeled ligand is brought in contact with the protein or its salt according to (1) or with the partial peptide or its salt according to (4), and (ii) when the labeled ligand and a test compound are brought in contact with the protein or its salt according to (1) or with the partial peptide or its salt according to (4); and comparing the amounts measured in (i) and (ii);

(26) A method of screening a compound or its salt that alters the binding property between a ligand and the protein or its salt according to (1), which comprises measuring the amounts of a labeled ligand bound to a cell containing the protein according to (1), (i) when the labeled ligand is brought in contact with the cell containing the protein according to (1), and (ii) when the labeled ligand and a test compound are brought in contact with the cell containing the protein according to (1); and comparing the amounts measured in (i) and (ii);

(27) A method of screening a compound or its salt that alters the binding property between a ligand and the protein or its salt according to (1), which comprises measuring the amounts of a labeled ligand bound to a cell membrane fraction containing the protein according to (1), (i) when the labeled ligand is brought in contact with the cell membrane fraction, and (ii) when the labeled ligand and a test compound are brought in contact with the cell membrane fraction; and comparing the amounts measured in (i) and (ii);

(28) A method of screening a compound or its salt that alters the binding property between a ligand and the protein or its salt according to (1), which comprises measuring the amounts of a labeled ligand bound to a protein expressed in a cell membrane, (i) when the labeled ligand is brought in contact with the protein expressed in a cell membrane of the transformant according to (9) by culturing the transformant and (ii) when the labeled ligand and a test compound are brought in contact with the protein expressed in a cell membrane of the transformant according to (9) by culturing the transformant; and comparing the amounts measured in (i) and (ii);

(29) A method of screening a compound or its salt that alters the binding property between a ligand and the protein or its salt according to (1), which comprises measuring the protein-mediated cell stimulating activities, (i) when a compound that activates the protein or its salt according to (1) is brought in contact with a cell containing the protein according to (1), and (ii) when a compound that activates the protein or its salt according to (1) and a test compound are brought in contact with a cell containing the protein according to (1); and comparing the activities measured in (i) and (ii);

(30) A method of screening a compound or its salt that alters the binding property between a ligand and the protein or its salt according to (1), which comprises measuring the protein-mediated cell stimulating activities, when a compound that activates the protein or its salt according to (1) is brought in contact with a G protein-coupled receptor protein expressed in a cell membrane of the transformant according to (9) by culturing the transformant, and when the compound that activates the protein or its salt according to (1) and a test compound are brought in contact with the protein expressed in a cell membrane of the transformant according to (9) by culturing the transformant; and comparing the protein-mediated activities measured in (i) and (ii);

(31) A method of screening according to (29) or (30), in which said compound that activates the protein according to (1) is angiotensin, bombesin, canavinoid, cholecystokinin, glutamine, serotonin, melatonin, neuropeptide Y, opioid, purine, vasopressin, oxytocin, PACAP, secretin, glucagon, calcitnonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotriene, pancreastatin, prostaglandin, thromboxane, adenosine, adrenaline, α- and β-chemokine (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide, galanin, MIT1 or their homologues derived from the mammal;

(32) A compound or its salt that alters the binding property between a ligand and the protein or its salt according to (1), which is obtainable by the screening methods according to (24) through (31);

(33) A pharmaceutical comprising a compound or its salt that alters the binding property between a ligand and the protein or its salt according to (1), which is obtainable by the screening methods according to (24) through (31);

(34) The kit for screening according to (14), comprising a cell containing the protein according to (1);

(35) The screening kit according to (14), comprising a cell membrane fraction containing the protein according to (1);

(36) The screening kit according to (14), comprising a protein expressed on the cell membrane of the transformant according to (9) by culturing the transformant;

(37) A compound or its salt that alters the binding property of a ligand and the protein or its salt according to (1), which is obtainable using the screening kits according to (34) through (36);

(38) A pharmaceutical comprising a compound or its salt that alters the binding property between a ligand and the protein or its salt according to (1), which is obtainable using the screening kits according to (34) through (36);

(39) A method of quantifying the protein according to (1), the partial peptide according to (4), or a salt thereof, which comprises contacting the antibody according to (11) with the protein according to (1), the partial peptide according to (4), or a salt thereof;

(40) A method of quantifying the protein according to (1), the partial peptide according to (4) or salts thereof in a test fluid, which comprises competitively reacting the antibody according to (11) with a test fluid and a labeled form of the protein according to (1), the partial peptide according to (4) or salts thereof; and measuring the ratios bound to the antibody of the labeled form of the protein according to (1), the partial peptide or its salts according to (4); and

(41) A method of quantifying the protein according to (1), the partial peptide according to (4), or salts thereof in a test fluid, which comprises reacting a test fluid simultaneously or sequentially with the antibody according to (11) immobilized on a carrier and the labeled antibody according to (11), and then measuring the activity of the label on the immobilizing carrier.

### BRIEF DESCRIPTION OF THE DRAWINGS

FIG. 1 shows a base sequence of DNA encoding rat brain-derived protein (rZAQ1) of the present invention obtained in EXAMPLE 1, and an amino acid sequence deduced therefrom (continued to FIG. 2).

FIG. 2 shows a base sequence of DNA encoding rat brain-derived protein (rZAQ1) of the present invention obtained in EXAMPLE 1, and an amino acid sequence deduced therefrom (continued from FIG. 1 and to FIG. 3).

FIG. 3 shows a base sequence of DNA encoding rat brain-derived protein (rZAQ1) of the present invention obtained in EXAMPLE 1, and an amino acid sequence deduced therefrom (continued from FIG. 2).

FIG. 4 shows a base sequence of DNA encoding rat brain-derived protein (rZAQ2) of the present invention obtained in EXAMPLE 2, and an amino acid sequence deduced therefrom (continued to FIG. 5).

FIG. 5 shows a base sequence of DNA encoding rat brain-derived protein (rZAQ2) of the present invention obtained in EXAMPLE 2, and an amino acid sequence deduced therefrom (continued from FIG. 5 and to FIG. 6).

FIG. 6 shows a base sequence of DNA encoding rat brain-derived protein (rZAQ2) of the present invention obtained in EXAMPLE 2, and an amino acid sequence deduced therefrom (continued from FIG. 5).

FIG. 7 shows a plot for hydrophobicity of rZAQ1.

FIG. 8 shows a plot for hydrophobicity of rZAQ2.

FIG. 9 shows a result of analysis for distribution of rZAQ1 expression.

FIG. 10 shows a result of analysis for distribution of rZAQ2 expression.

### BEST MODE FOR CARRYING OUT THE INVENTION

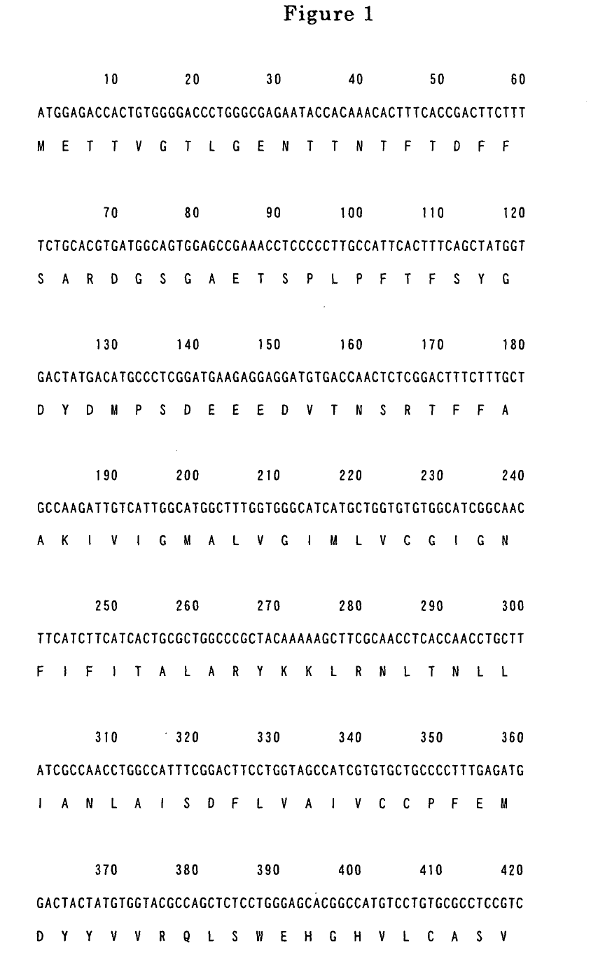

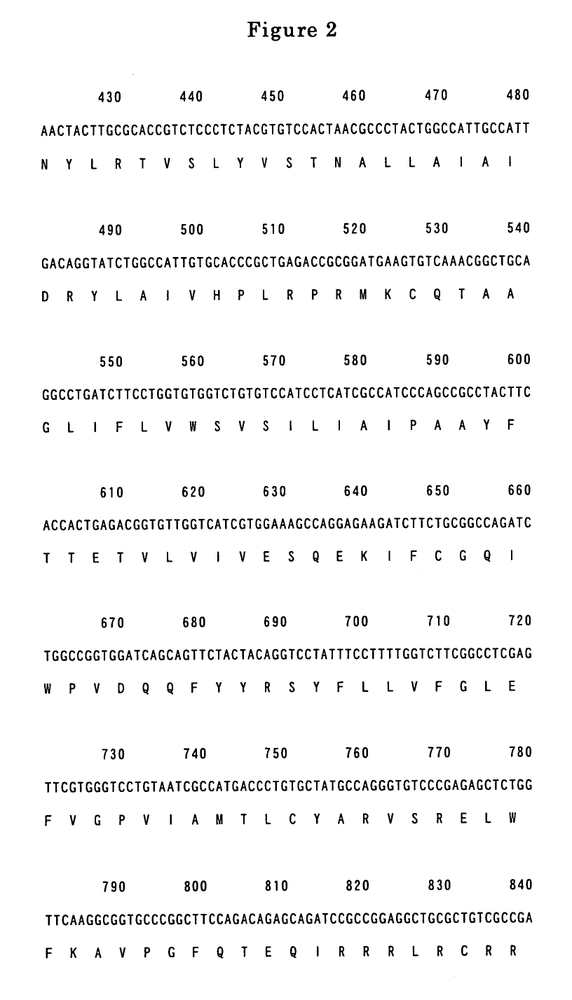

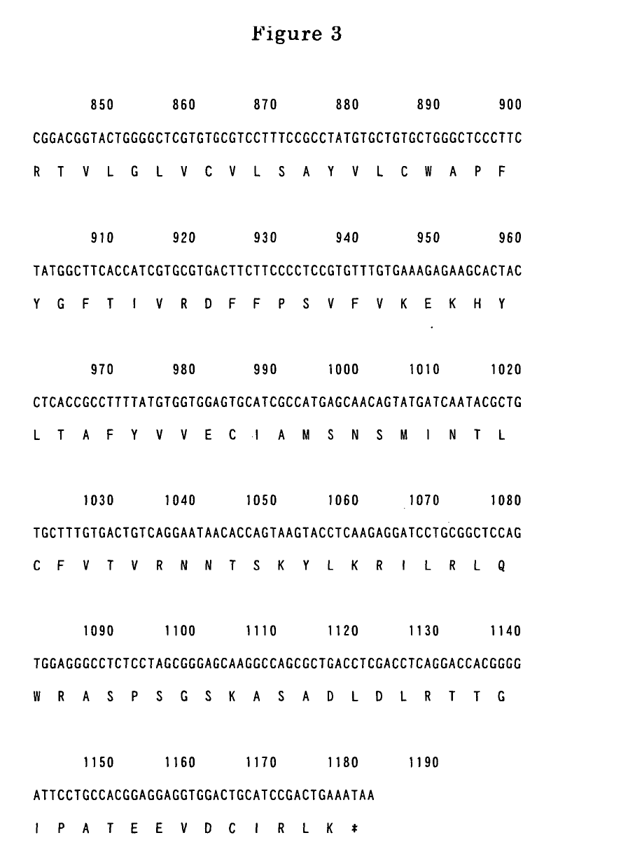

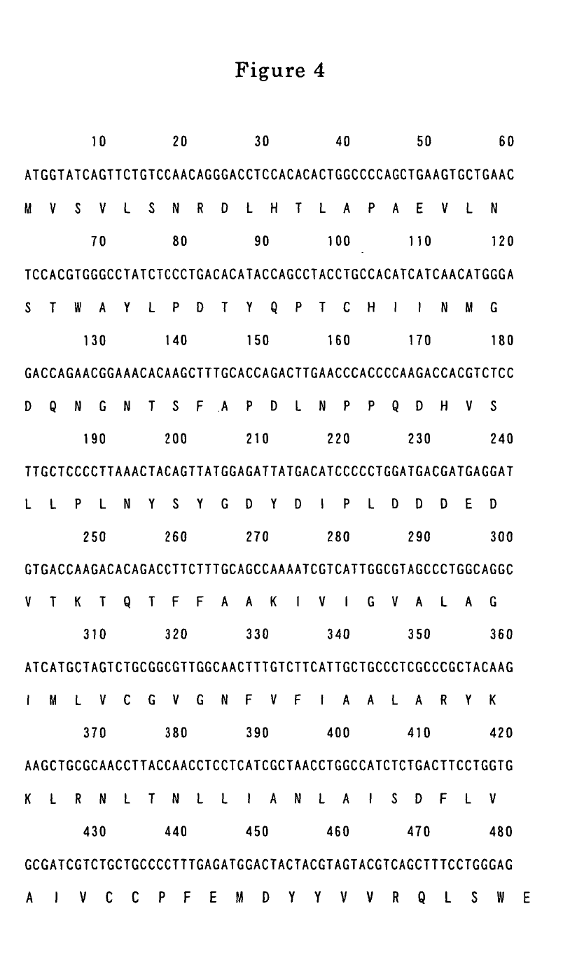

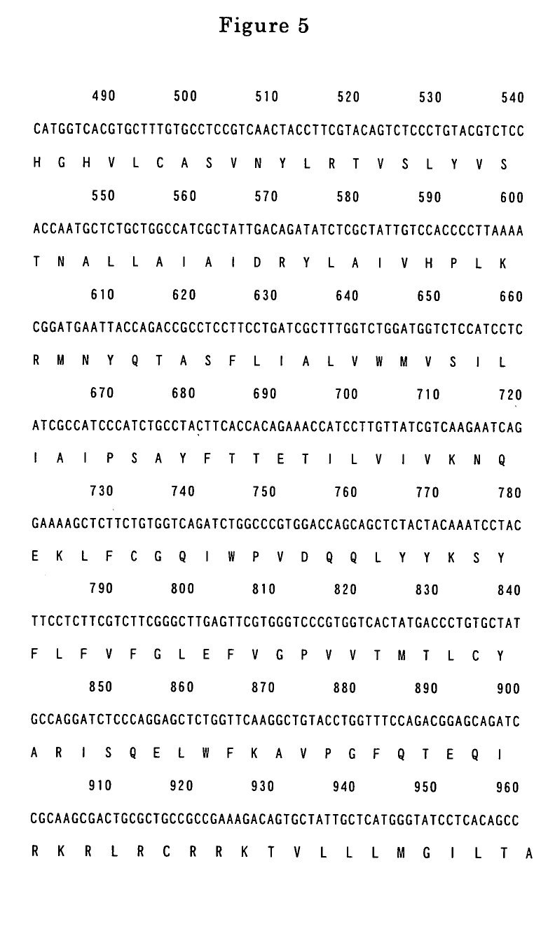

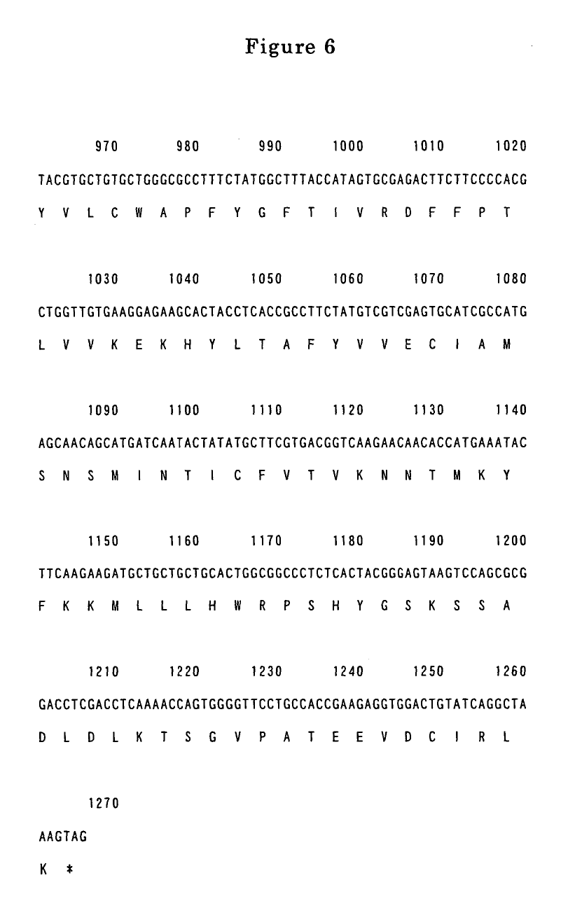

The protein (the G protein-coupled receptor protein) of the present invention is a receptor protein, which contains the same or substantially the same amino acid sequence as the amino acid sequence shown by SEQ ID NO: 4 (the amino acid sequence from FIG. 1 to FIG. 3) or SEQ ID NO: 11 (the amino acid sequence from FIG. 4 to FIG. 6) (hereinafter sometimes merely referred the protein (the G protein-coupled receptor protein) of the present invention or its salt to as the protein of the present invention).

The protein (the G protein-coupled receptor protein) of the present invention may be any protein derived from any cells (e.g., retina cells, liver cells, splenocytes, nerve cells, glial cells, β cells of pancreas, bone marrow cells, mesangial cells, Langerhans' cells, epidermic cells, epithelial cells, endothelial cells, fibroblasts, fibrocytes, myocytes, fat cells, immune cells (e.g., macrophage, T cells, B cells, natural killer cells, mast cells, neutrophil, basophil, eosinophil, monocyte), megakaryocyte, synovial cells, chondrocytes, bone cells, osteoblasts, osteoclasts, mammary gland cells, hepatocytes or interstitial cells, the corresponding precursor cells, stem cells, cancer cells, etc.), hemocyte type cells (e.g., MEL, M1, CTLL-2, HT-2, WEHI-3, HL-60, JOSK-1, K562, ML-1, MOLT-1, MOLT-4, MOLT-10, CCRF-CEM, TALL-1, Jurkat, CCRT-HSB-2, KE-37, SKW-3, HUT-78, HUT-102, H9, U937, THP-1, HEL, JK-1, CMK, KO-812, MEG-01, etc.), or any tissues where such cells are present, e.g., brain or any region of the brain (e.g., olfactory bulb, amygdaloid nucleus, basal ganglia, hippocampus, thalamus, hypothalamus, subthalamic nucleus, cerebral cortex, medulla oblongata, cerebellum, occipital pole, frontal lobe, temporal lobe, putamen, caudate nucleus, corpus callosum, substantia nigra), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, peripheral blood cells, prostate, testis, ovary, placenta, uterus, bone, joint, skeletal muscle, etc. (in particular, brain or any region of the brain) from human and other mammals (e.g., guinea pigs, rats, mice, rabbits, swine, sheep, bovine, monkeys, etc.). The receptor protein may also be a synthetic protein.

The amino acid sequence which has substantially the same amino acid sequence as that represented by SEQ ID NO: 4 includes an amino acid sequence having at least about 97% homology, preferably at least about 98% homology, more preferably at least about 99% homology, and the most preferably at least about 99.5% homology, to the amino acid sequence represented by SEQ ID NO: 4.

Examples of the protein having substantially the same amino acid sequence as that shown by SEQ ID NO: 4 include a protein having substantially the same amino acid sequence as that shown by SEQ ID NO: 4 and having the nature substantially equivalent to the protein having the amino acid sequence represented by SEQ ID NO: 4, etc.

Preferred examples of the protein containing the same or substantially the same amino acid sequence as that shown by SEQ ID NO: 4 of the present invention include a protein containing the same or substantially the same amino acid sequence as that shown by SEQ ID NO: 4 and having the activity substantially equivalent to the amino acid sequence represented by SEQ ID NO: 4, etc.

The amino acid sequence which has substantially the same amino acid sequence as that represented by SEQ ID NO: 11 includes an amino acid sequence having at least about 95% homology, preferably at least about 96% homology, more preferably at least about 97% homology, and the most preferably at least about 98% homology, to the amino acid sequence represented by SEQ ID NO: 11.

Examples of the protein having substantially the same amino acid sequence as that shown by SEQ ID NO: 11 include a protein having substantially the same amino acid sequence as that shown by SEQ ID NO: 11 and having the nature substantially equivalent to the protein having the amino acid sequence represented by SEQ ID NO: 11, etc.

Preferred examples of the protein containing the same or substantially the same amino acid sequence as that shown by SEQ ID NO: 11 of the present invention include a protein containing the same or substantially the same amino acid sequence as that shown by SEQ ID NO: 11 and having the activity substantially equivalent to the amino acid sequence represented by SEQ ID NO: 11, etc.

Examples of the substantially equivalent activity include a ligand binding activity, a signal transduction activity, etc. The term “substantially equivalent” is used to mean that the nature of the activity is the same. Therefore, although it is preferred those activities such as the ligand binding and signal transduction activities, etc. are equivalent (e.g., about 0.5- to about 2-fold), quantitative factors such as a level of the activity, a molecular weight of the protein, etc. may differ.

The activities such as ligand binding and signal transduction activities or the like can be determined according to a publicly known method with some modifications, for example, by the ligand determination methods or the screening methods.

Proteins containing the following amino acid sequences are used as the receptor protein of the present invention: \mathring{1} amino acid sequences represented by SEQ ID NO: 4 or SEQ ID NO: 11, wherein at least 1 or 2 amino acids (preferably approximately 1 to 30 amino acids, more preferably approximately 1 to 10 amino acids, most preferably several (1 to 2) amino acids) are deleted; \mathring{2} amino acid sequences represented by SEQ ID NO: 4 or SEQ ID NO: 11, to which at least 1 or 2 amino acids (preferably approximately 1 to 30 amino acids, more preferably approximately 1 to 10 amino acids, and most preferably several (1 to 2) amino acids) are added; \mathring{3} amino acid sequences represented by SEQ ID NO: 4 or SEQ ID NO: 11, in which at least 1 or 2 amino acids (preferably approximately 1 to 30 amino acids, more preferably approximately 1 to 10 amino acids, and most preferably several (1 to 2) amino acids) are substituted by other amino acids; or \mathring{4} combination of the amino acid sequences described in the above.

Throughout the present specification, the proteins are represented in accordance with the conventional way of describing peptides, that is, the N-terminus (amino terminus) at the left hand and the C-terminus (carboxyl terminus) at the right hand. In the proteins of the present invention including the proteins containing the amino acid sequence shown by SEQ ID NO: 4 or SEQ ID NO: 11, the C-terminus is usually in the form of a carboxyl group (—COOH) or a carboxylate (—COO⁻) but may be in the form of an amide (—CONH₂) or an ester (—COOR).

Examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl group such as a phenyl-C₁₋₂-alkyl group, e.g., benzyl, phenethyl, etc., or an α-naphthyl-C₁₋₂-alkyl group such as α-naphthylmethyl, etc.; and the like. In addition, pivaloyloxymethyl or the like, which is used widely as an ester for oral administration, may also be used.

Where the protein of the present invention contains a carboxyl group (or a carboxylate) at a position other than the C-terminus, it may be amidated or esterified and such an amide or ester is also included within the protein of the present invention. The ester group may be the same group as that described with respect to the C-terminus described above.

Furthermore, examples of the protein of the present invention include variants of the above receptor proteins, wherein the amino group at the N-terminal methionine residue of the protein supra is protected with a protecting group (for example, a C₁₋₆ acyl group such as a C₂₋₆ alkanoyl group, e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₂₋₆ alkanoyl group, e.g., formyl group, acetyl group, etc.), or conjugated proteins such as glycoproteins bound to sugar chains.

Specific examples of the protein of the present invention which can be used include a rat-derived (more preferably rat brain-derived) protein containing an amino acid sequence represented by SEQ ID NO: 4 or SEQ ID NO: 11, etc.

As partial peptides of the protein of the present invention (hereinafter sometimes referred to as the partial peptides of the present invention, or simply the partial peptides), any partial peptide can be used so long as it can be a partial peptide of the protein. Among the protein molecules of the present invention, for example, those having a site exposed to the outside of a cell membrane and having a receptor binding activity can be used.

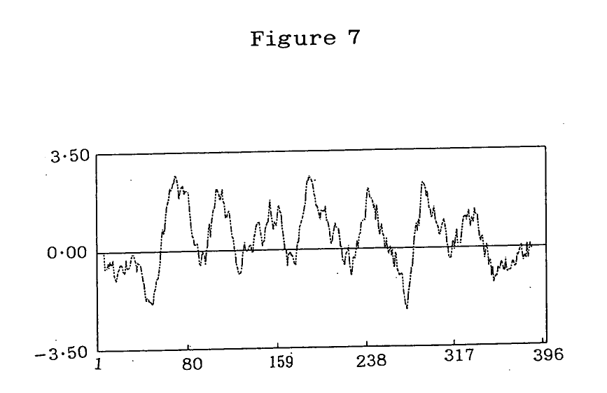

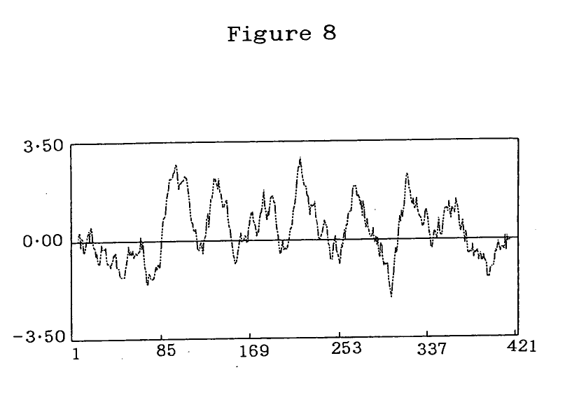

Specifically, the partial peptide of the protein having the amino acid sequence represented by SEQ ID NO: 4 or SEQ ID NO: 11 is a peptide containing the parts analyzed to be extracellular domains (hydrophilic domains) in the hydrophobic plotting analysis shown in FIG. 7 or FIG. 8. A peptide containing a hydrophobic domain in part can be used as well. In addition, the peptide may contain each domain separately or plural domains together.

In the protein of the present invention, preferred partial peptides are those having at least 20, preferably at least 50, and more preferably at least 100 amino acids, in the amino acid sequence which constitutes the protein of the present invention.

The amino acid sequence having substantially the same amino acid sequence includes an amino acid sequence having at least about 50% homology, preferably at least about 70% homology, more preferably at least about 80% homology, much more preferably at least about 90% homology, and most preferably at least about 95% homology, to these amino acid sequences.

Herein, the term “activity substantially equivalent” refers to the same significance as defined above. The “activity substantially equivalent” can be assayed in the same manner as given above.

The partial peptide of the present invention may contain an amino acid sequence, wherein at least 1 or 2 amino acids (preferably approximately 1 to 10 amino acids, more preferably several (1 to 2) amino acids) are deleted; to which at least 1 or 2 amino acids (preferably approximately 1 to 20 amino acids, more preferably approximately 1 to 10 amino acids, and most preferably several (1 to 2) amino acids) are added; or, in which at least 1 or 2 amino acids (preferably approximately 1 to 10 amino acids, more preferably several and most preferably approximately 1 to 2 amino acids) are substituted by other amino acids.

In the partial peptide of the present invention, the C-terminus is normally a carboxyl group (—COOH) or carboxylate (—COO⁻) but the C-terminus may be in the form of an amide (—CONH₂) or an ester (—COOR), as has been described with the protein of the present invention.

As in the protein of the present invention described above, the partial peptide of the present invention further includes those in which the amino group of the amino acid residue of the N-terminal methionine residue is protected by a protecting group, those in which the N-terminal residue is cleaved in vivo and the produced glutamine residue is pyroglutaminated, those in which substituents on the side chains of amino acids in the molecule are protected by appropriate protecting groups, conjugated peptides such as so-called glycoproteins, to which sugar chains are bound, and the like.

In the partial peptide of the present invention, the C-terminus is normally a carboxyl group (—COOH) or carboxylate (—COO⁻) but the C-terminus may be in the form of an amide (—CONH₂) or an ester (—COOR), as has been described with the protein of the present invention.

For salts of the protein or the partial peptide of the present invention, preferred are salts with especially physiologically acceptable acid addition salts. Examples of the salts include salts with, for example, inorganic acids (e.g., hydrochloric acid, phosphoric acid, hydrobromic acid, sulfuric acid); salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The protein of the present invention or salts thereof may be manufactured by a publicly known method used to purify a protein from human or other mammalian cells or tissues described above, or by culturing a transformant that contains the DNA encoding the protein of the present invention, as will be later described. Furthermore, the protein or its salts may also be manufactured by the methods for synthesizing proteins or by modifications thereof, which will also be described hereinafter.

Where the protein or its salts are manufactured from human or mammalian tissues or cells, human or mammalian tissues or cells are homogenized, then extracted with an acid or the like, and the extract is isolated and purified by a combination of chromatography techniques such as reverse phase chromatography, ion exchange chromatography, and the like.

To synthesize the protein of the present invention, its partial peptide, or salts or amides thereof according to the present invention, commercially available resins that are used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmehtylphenyl acetamidomethyl resin, polyacrylamide resin, 4-(2′,4′-dimethoxyphenylhydroxymethyl)phenoxy resin, 4-(2′,4′-dimethoxyphenyl-Fmoc-aminoethyl)phenoxy resin, etc. Using these resins, amino acids in which -amino groups and functional groups on the side chains are appropriately protected are condensed on the resin in the order of the sequence of the objective protein according to various condensation methods publicly known in the art. At the end of the reaction, the receptor protein is cut out from the resin and at the same time, the protecting groups are removed. Then, intramolecular disulfide bond-forming reaction is performed in a highly diluted solution to obtain the objective protein or its amides.

For condensation of the protected amino acids described above, a variety of activation reagents for protein synthesis may be used, and carbodiimides are particularly preferable. Examples of such carbodiimides include DCC, N,N′-diisopropylcarbodiimide, N-ethyl-N′-(3-dimethylaminoprolyl)carbodiimide, etc. For activation by these reagents, the protected amino acids in combination with a racemization inhibitor (e.g., HOBt, HOOBt) are added directly to the resin, or the protected amino acids are previously activated in the form of symmetric acid anhydrides, HOBt esters or HOOBt esters, followed by adding the thus activated protected amino acids to the resin.

Solvents suitable for use to activate the protected amino acids or condense with the resin may be chosen from solvents known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitrites such as acetonitrile, propionitrile, etc.; esters such as methyl acetate, ethyl acetate, etc.; and appropriate mixtures of these solvents. The reaction temperature is appropriately chosen from the range known to be applicable to protein binding reactions and is usually selected in the range of approximately −20° C. to 50° C. The activated amino acid derivatives are used generally in an excess of 1.5 to 4 times. The condensation is examined by a test using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole.

Examples of the protecting groups used to protect the amino groups of the starting compounds include Z, Boc, t-pentyloxycarbonyl, isobornyloxycarbonyl, 4-methoxybenzyloxycarbonyl, Cl—Z, Br—Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (in the form of linear, branched or cyclic alkyl esters of the alkyl moiety such as methyl, ethyl, propyl, butyl, t-butyl, cyclopentyl, cyclohexyl, cycloheptyl, cyclooctyl, 2-adamantyl, etc.), aralkyl esterification (e.g., esterification in the form of benzyl ester, 4-nitrobenzyl ester, 4-methoxybenzyl ester, 4-chlorobenzyl ester, benzhydryl ester, etc.), phenacyl esterification, benzyloxycarbonyl hydrazidation, t-butoxycarbonyl hydrazidation, trityl hydrazidation, or the like.

The hydroxyl group of serine can be protected through, for example, its esterification or etherification. Examples of groups appropriately used for the esterification include a lower alkanoyl group, such as acetyl group, an aroyl group such as benzoyl group, and a group derived from carbonic acid such as benzyloxycarbonyl group, ethoxycarbonyl group, etc. Examples of a group appropriately used for the etherification include benzyl group, tetrahydropyranyl group, t-butyl group, etc.

Examples of groups for protecting the phenolic hydroxyl group of tyrosine include Bzl, Cl₂—Bzl, 2-nitrobenzyl, Br—Z, t-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting compounds include the corresponding acid anhydrides, azides, activated esters (esters with alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)). As the activated amino acids, in which the amino groups are activated in the starting material, the corresponding phosphoric amides are employed.

To eliminate (split off) the protecting groups, there are used catalytic reduction under hydrogen gas flow in the presence of a catalyst such as Pd-black or Pd-carbon; an acid treatment with anhydrous hydrogen fluoride, methanesulfonic acid, trifluoromethanesulfonic acid or trifluoroacetic acid, or a mixture solution of these acids; a treatment with a base such as diisopropylethylamine, triethylamine, piperidine or piperazine; and reduction with sodium in liquid ammonia. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol or 1,2-ethanedithiol. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol or 1,4-butanedithiol, as well as by a treatment with an alkali such as a dilute sodium hydroxide solution and dilute ammonia.

Protection of functional groups that should not be involved in the reaction of the starting materials, protecting groups, elimination of the protecting groups and activation of functional groups involved in the reaction may be appropriately selected from publicly known groups and publicly known means.

In another method for obtaining the amides of the protein, for example, the α-carboxyl group of the carboxy terminal amino acid is first protected by amidation; the peptide (protein) chain is then extended from the amino group side to a desired length. Thereafter, a protein in which only the protecting group of the N-terminal α-amino group in the peptide chain has been eliminated from the protein and a protein in which only the protecting group of the C-terminal carboxyl group has been eliminated are prepared. The two proteins are condensed in a mixture of the solvents described above. The details of the condensation reaction are the same as described above. After the protected protein obtained by the condensation is purified, all the protecting groups are eliminated by the method described above to give the desired crude protein. This crude protein is purified by various known purification means. Lyophilization of the major fraction gives the amide of the desired protein.

To prepare the esterified protein, for example, the α-carboxyl group of the carboxy terminal amino acid is condensed with a desired alcohol to prepare the amino acid ester, which is followed by procedure similar to the preparation of the amidated protein above to give the ester form of the desired protein.

The partial peptide or its salts in the protein of the present invention can be manufactured by publicly known methods for peptide synthesis, or by cleaving the protein of the present invention with an appropriate peptidase. For the methods for peptide synthesis, for example, either solid phase synthesis or liquid phase synthesis may be used. That is, the partial peptide or amino acids that can construct the protein of the present invention are condensed with the remaining part. Where the product contains protecting groups, these protecting groups are removed to give the desired peptide. Publicly known methods for condensation and elimination of the protecting groups are described in \mathring{1}-\mathring{5} below.

\mathring{1} M. Bodanszky & M. A. Ondetti: Peptide Synthesis, Interscience Publishers, New York (1966)

\mathring{2} Schroeder & Luebke: The Peptide, Academic Press, New York (1965)

\mathring{3} Nobuo Izumiya, et al.: Peptide Gosei-no-Kiso to Jikken (Basics and experiments of peptide-synthesis), published by Maruzen Co. (1975)

\mathring{4} Haruaki Yajima & Shunpei Sakakibara: Seikagaku Jikken Koza (Biochemical Experiment) 1, Tanpakushitsu no Kagaku (Chemistry of Proteins) IV, 205 (1977)

\mathring{5} Haruaki Yajima, ed.: Zoku Iyakuhin no Kaihatsu (A sequel to Development of Pharmaceuticals), Vol. 14, Peptide Synthesis, published by Hirokawa Shoten

After completion of the reaction, the product may be purified and isolated by a combination of conventional purification methods such as solvent extraction, distillation, column chromatography, liquid chromatography and recrystallization to give the partial peptide of the present invention. When the partial peptide obtained by the above methods is in a free form, the peptide can be converted into an appropriate salt by a publicly known method; when the protein is obtained in a salt form, it can be converted into a free form by a publicly known method.

The DNA encoding the protein of the present invention may be any DNA so long as it contains the base sequence encoding the protein of the present invention described above. The DNA encoding the protein of the present invention may be any of genomic DNA, genomic DNA library, cDNA derived from the cells and tissues described above, cDNA library derived from the cells and tissues described above and synthetic DNA. The vector to be used for the library may be any of bacteriophage, plasmid, cosmid and phagemid. The DNA may also be directly amplified by reverse transcriptase polymerase chain reaction (hereinafter abbreviated as RT-PCR) using the total RNA or mRNA fraction prepared from the cells and tissues described above.

Specifically, the DNA encoding the protein of the present invention may be any DNA containing the base sequence shown by SEQ ID NO: 3 or SEQ ID NO: 10, or having the DNA, which hybridizes to the DNA having the base sequence represented by SEQ ID NO: 3 or SEQ ID NO: 10 under highly stringent conditions and encoding a protein having the activities substantially equivalent to those of the protein of the present invention (e.g., a ligand binding activity, a signal transduction activity, etc.).

Specific examples of the DNA hybridizable to the base sequence represented by SEQ ID NO: 3 under highly stringent conditions include DNA containing a base sequence having at least about 97% homology, preferably at least about 98% homology, more preferably at least about 99% homology and the most preferably at least about 99.5% homology, to the base sequence represented by SEQ ID NO: 3.

Specific examples of the DNA hybridizable to the base sequence represented by SEQ ID NO: 10 under highly stringent conditions include DNA containing a base sequence having at least about 95% homology, preferably at least about 96% homology, more preferably at least about 97% homology and the most preferably at least about 98% homology, to the base sequence represented by SEQ ID NO: 10.

The hybridization can be carried out by publicly known methods or by modifications of these methods, for example, according to the method described in Molecular Cloning, 2nd (J. Sambrook et al., Cold Spring Harbor Lab. Press, 1989). A commercially available library may also be used according to the instructions of the attached manufacturer's protocol. Preferably, the hybridization can be carried out under highly stringent conditions.

The highly stringent conditions used herein are, for example, those in a sodium concentration at about 19 mM to about 40 mM, preferably about 19 mM to about 20 mM at a temperature of about 50° C. to about 70° C., preferably about 60° C. to about 65° C. In particular, hybridization conditions in a sodium concentration of about 19 mM at a temperature of about 65° C. are most preferred.

More specifically, for the DNA encoding the protein having the amino acid sequence represented by SEQ ID NO: 4, there may be included DNA having the base sequence represented by SEQ ID NO: 3. Further, for the DNA encoding the protein having the amino acid sequence represented by SEQ ID NO: 11, there may be included DNA having the base sequence represented by SEQ ID NO: 10.

The nucleotide (oligonucleotide) comprising the base sequence encoding the protein of the present invention or a part of the base sequence complementary to the base sequence is used to mean to embrace not only the DNA encoding the protein or its partial peptide of the present invention but also RNA.

According to the present invention, antisense (oligo)nucleotides (nucleic acids) that can inhibit the replication or expression of protein genes can be designed and synthesized based on the base sequence information of the cloned or determined DNA encoding the protein. Such a (oligo)nucleotide (nucleic acid) is capable of hybridizing to RNA of G protein-coupled protein gene to inhibit the synthesis or function of said RNA or capable of modulating or controlling the expression of a G protein-coupled protein gene via interaction with G protein-coupled protein-associated RNA. (Oligo)nucleotides complementary to the selected sequences of RNA associated with G protein-coupled protein and (oligo)nucleotides specifically hybridizable to the G protein-coupled protein-associated RNA are useful in modulating or controlling the expression of a G protein-coupled protein gene in vivo and in vitro, and useful for the treatment or diagnosis of diseases.

The term “corresponding” is used to mean homologous to or complementary to a particular sequence of the nucleotide, base sequence or nucleic acid including the gene. The term “corresponding” between nucleotides, base sequences or nucleic acids and peptides (proteins) usually refer to amino acids of a peptide (protein) under the order derived from the sequence of nucleotides (nucleic acids) or their complements. In the G protein-coupled protein genes, the 5′ end hairpin loop, 5′ end 6-base-pair repeats, 5′ end untranslated region, polypeptide translation initiation codon, protein coding region, ORF translation initiation codon, 3′ end untranslated region, 3′ end palindrome region, and 3′ end hairpin loop, may be selected as preferred target regions, though any other region may be selected as a target in the G protein-coupled protein genes.

The relationship between the targeted nucleic acids and the (oligo)nucleotides complementary to at least a part of the target, specifically the relationship between the target and the (oligo)nucleotides hybridizable to the target, can be denoted to be “antisense”. Examples of the antisense polynucleotides include polydeoxynucleotides containing 2-deoxy-D-ribose, polynucleotides containing D-ribose, any other type of polynucleotides which are N-glycosides of a purine or pyrimidine base, or other polymers containing non-nucleotide backbones (e.g., protein nucleic acids and synthetic sequence-specific nucleic acid polymers commercially available) or other polymers containing nonstandard linkages (provided that the polymers contain nucleotides having such a configuration that allows base pairing or base stacking, as is found in DNA or RNA), etc. The antisense polynucleotides may be double-stranded DNA, single-stranded DNA, single-stranded RNA or a DNA:RNA hybrid, and may further include unmodified polynucleotides (or unmodified oligonucleotides), those with publicly known types of modifications, for example, those with labels known in the art, those with caps, methylated polynucleotides, those with substitution of one or more naturally occurring nucleotides by their analogue, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (nucleases, nuclease inhibitors, toxins, antibodies, signal peptides, poly-L-lysine, etc.), saccharides (e.g., monosaccharides, etc.), those with intercalators (e.g., acridine, psoralen, etc.), those containing chelators (e.g., metals, radioactive metals, boron, oxidative metals, etc.), those containing alkylating agents, those with modified linkages (e.g., a anomeric nucleic acids, etc.), and the like. Herein the terms “nucleoside”, “nucleotide” and “nucleic acid” are used to refer to moieties that contain not only the purine and pyrimidine bases, but also other heterocyclic bases, which have been modified. Such modifications may include methylated purines and pyrimidines, acylated purines and pyrimidines and other heterocyclic rings. Modified nucleotides and modified nucleotides also include modifications on the sugar moiety, wherein, for example, one or more hydroxyl groups may optionally be substituted with a halogen atom(s), an aliphatic group(s), etc., or may be converted into the corresponding functional groups such as ethers, amines, or the like.

The antisense nucleic acid of the present invention is RNA, DNA or a modified nucleic acid. Specific examples of the modified nucleic acid are, but not limited to, sulfur and thiophosphate derivatives of nucleic acids and those resistant to degradation of polynucleoside amides or oligonucleoside amides. The antisense nucleic acids of the present invention can be modified preferably based on the following design, that is, by increasing the intracellular stability of the antisense nucleic acid, increasing the cellular permeability of the antisense nucleic acid, increasing the affinity of the nucleic acid to the targeted sense strand to a higher level, or minimizing the toxicity, if any, of the antisense nucleic acid.

Many of such modifications are known in the art, as disclosed in J. Kawakami, et al., Pharm. Tech. Japan, Vol. 8, pp. 247, 1992; Vol. 8, pp. 395, 1992; S. T. Crooke, et al. ed., Antisense Research and Applications, CRC Press, 1993; etc.

The antisense nucleic acid of the present invention may contain altered or modified sugars, bases or linkages. The antisense nucleic acid may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached to the nucleic acid at the 3′ or 5′ ends thereof and may also be attached thereto through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include, but are not limited to, hydroxyl protecting groups known in the art, including glycols such as polyethylene glycol, tetraethylene glycol and the like.

The inhibitory action of the antisense nucleic acid can be examined using the transformant of the present invention, the gene expression system of the present invention in vivo and in vitro, or the translation system of the protein in vivo and in vitro. The nucleic acid can be applied to cells by a variety of publicly known methods.

The DNA encoding the partial peptide of the present invention may be any DNA so long as it contains the base sequence encoding the partial peptide of the present invention described above. The DNA may also be any of genomic DNA, genomic DNA library, cDNA derived from the cells and tissues described above, cDNA library derived from the cells and tissues described above and synthetic DNA. The vector to be used for the library may be any of bacteriophage, plasmid, cosmid and phagemid. The DNA may also be directly amplified by RT-PCR method using mRNA fraction prepared from the cells and tissues described above.

Specifically, the DNA encoding the partial peptide of the present invention may be any one of, for example, (1) DNA having a partial base sequence of the DNA having the base sequence represented by SEQ ID NO: 3 or SEQ ID NO: 10, or (2) any DNA having a partial base sequence of the DNA having a base sequence hybridizable to the base sequence represented by SEQ ID NO: 3 or SEQ ID NO: 10 under highly stringent conditions and encoding a protein which has the activities (e.g., a ligand-biding activity, a signal transduction activity, etc.) substantially equivalent to those of the protein peptide of the present invention.

Specific examples of the DNA that hybridizes to the DNA having the base sequence represented by SEQ ID NO: 3 under highly stringent conditions include DNA containing a base sequence having at least about 97% homology, preferably at least about 98% homology, more preferably at least about 99% homology and the most preferably at least about 99.5% homology, to the base sequence represented by SEQ ID NO: 3.

Specific examples of the DNA that hybridizes to the DNA having the base sequence represented by SEQ ID NO: 10 under highly stringent conditions include DNA containing a base sequence having at least about 95% homology, preferably at least about 96% homology, more preferably at least about 97% homology and the most preferably at least about 98% homology, to the base sequence represented by SEQ ID NO: 10.

For cloning of the DNA that completely encodes the protein of the present invention or its partial peptide (hereinafter sometimes collectively referred to as the protein of the present invention), the DNA may be either amplified by PCR using synthetic DNA primers having a part of the base sequence of the DNA encoding the protein of the present invention, or the DNA inserted into an appropriate vector can be selected by hybridization with a labeled DNA fragment or synthetic DNA that encodes a part or entire region of the protein of the present invention. The hybridization can be carried out, for example, according to the method described in Molecular Cloning, 2nd, J. Sambrook et al., Cold Spring Harbor Lab. Press, 1989. The hybridization may also be performed using commercially available library in accordance with the protocol described in the attached instructions.

Conversion of the base sequence of the DNA can be effected by publicly known methods such as the ODA-LA PCR method, the Gupped duplex method or the Kunkel method or its modification by using a publicly known kit available as Mutan™-super Express (Takara Shuzo Co., Ltd.) or Mutan™-K (Takara Shuzo Co., Ltd.).

The cloned DNA encoding the protein can be used as it is, depending upon purpose or, if desired, after digestion with a restriction enzyme or after addition of a linker thereto. The DNA may contain ATG as a translation initiation codon at the 5′ end thereof and may further contain TAA, TGA or TAG as a translation termination codon at the 3′ end thereof. These translation initiation and termination codons may also be added by using an appropriate synthetic DNA adapter.

The expression vector for the protein of the present invention can be manufactured, for example, by (a) excising the desired DNA fragment from the DNA encoding the protein of the present invention, and then (b) ligating the DNA fragment with an appropriate expression vector downstream a promoter in the vector.

Examples of the vector include plasmids derived from E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNAI/Neo, pcDNA3.1, pRc/CMV2, pRc/RSV (Invitrogen), etc.

The promoter used in the present invention may be any promoter if it matches well with a host to be used for gene expression. In the case of using animal cells as the host, examples of the promoter include SRα promoter, SV40 promoter, HIV-LTR promoter, CMV promoter, HSV-TK promoter, etc.

Among them, CMV promoter or SRα promoter is preferably used. Where the host is bacteria of the genus Escherichia, preferred examples of the promoter include trp promoter, lac promoter, recA promoter, λP_{L} promoter, lpp promoter, etc. In the case of using bacteria of the genus Bacillus as the host, preferred example of the promoter are SPO1 promoter, SPO2 promoter and penP promoter. When yeast is used as the host, preferred examples of the promoter are PHO5 promoter, PGK promoter, GAP promoter and ADH promoter. When insect cells are used as the host, preferred examples of the promoter include polyhedrin prompter and P10 promoter.

In addition to the foregoing examples, the expression vector may further optionally contain an enhancer, a splicing signal, a polyA addition signal, a selection marker, SV40 replication origin (hereinafter sometimes abbreviated as SV40ori) etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. In particular, when dhfr gene is used as the selection marker in dhfr gene defective Chinese hamster cells CHO (hereinafter referred to as CHO (dhfr⁻) cells), selection can also be made on thymidine free media.

If necessary and desired, a signal sequence that matches with a host is added to the N-terminus of the protein of the present invention. Examples of the signal sequence that can be used are Pho A signal sequence, OmpA signal sequence, etc. in the case of using bacteria of the genus Escherichia as the host; α-amylase signal sequence, subtilisin signal sequence, etc. in the case of using bacteria of the genus Bacillus as the host; MFα signal sequence, SUC2 signal sequence, etc. in the case of using yeast as the host; and insulin signal sequence, α-interferon signal sequence, antibody molecule signal sequence, etc. in the case of using animal cells as the host, respectively.

Using the vector containing the DNA encoding the protein of the present invention thus constructed, transformants can be manufactured.

Examples of the host, which may be employed, are bacteria belonging to the genus Escherichia, bacteria belonging to the genus Bacillus, yeast, insect cells, insects and animal cells, etc.

Specific examples of the bacteria belonging to the genus Escherichia include Escherichia coli K12 DH1 (Proc. Natl. Acad. Sci. U.S.A., 60, 160 (1968)), JM103 (Nucleic Acids Research, 9, 309 (1981)), JA221 (Journal of Molecular Biology, 120, 517 (1978)), HB101 (Journal of Molecular Biology, 41, 459 (1969)), C600 (Genetics, 39, 440 (1954)), etc.

Examples of the bacteria belonging to the genus Bacillus include Bacillus subtilis MI114 (Gene, 24, 255 (1983)), 207-21 (Journal of Biochemistry, 95, 87 (1984)), etc.

Examples of yeast include Saccharomyces cereviseae AH22, AH22R⁻, NA87-11A, DKD-5D, 20B-12, Schizosaccharomyces pombe NCYC1913, NCYC2036, Pichia pastoris KM71, etc.

Examples of insect cells include, for the virus AcNPV, Spodoptera frugiperda cells (Sf cells), MG1 cells derived from mid-intestine of Trichoplusia ni, High Five™ T cells derived from egg of Trichoplusia ni, cells derived from Mamestra brassicae, cells derived from Estigmena acrea, etc.; and for the virus BmNPV, Bombyx mori N cells (BmN cells), etc. are used. Examples of the Sf cell which can be used are Sf9 cells (ATCC CRL1711) and Sf21 cells (both cells are described in Vaughn, J. L. et al., In Vivo, 13, 213-217 (1977).

As the insect, for example, a larva of Bombyx mori can be used (Maeda, et al., Nature, 315, 592 (1985)).

Examples of animal cells include monkey cells COS7, Vero, Chinese hamster cells CHO (hereinafter referred to as CHO cells), CHO(dhfr⁻) cell, mouse L cells, mouse AtT-20, mouse myeloma cells, rat GH3, human FL cells, etc.

Bacteria belonging to the genus Escherichia can be transformed, for example, by the method described in Proc. Natl. Acad. Sci. U.S.A., 69, 2110 (1972) or Gene, 17, 107 (1982).

Bacteria belonging to the genus Bacillus can be transformed, for example, by the method described in Molecular & General Genetics, 168, 111 (1979).

Yeast can be transformed, for example, by the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55(1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995), published by Shujunsha, or Virology, 52, 456 (1973).

Thus, the transformant transformed with the expression vector containing the DNA encoding the G protein-coupled protein can be obtained.

Where the host is bacteria belonging to the genus Escherichia or the genus Bacillus, the transformant can be appropriately incubated in a liquid medium which contains materials required for growth of the transformant such as carbon sources, nitrogen sources, inorganic materials, and so on. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast extract, vitamins, growth promoting factors etc. may also be added to the medium. Preferably, pH of the medium is adjusted to about 5 to about 8.

A preferred example of the medium for incubation of the bacteria belonging to the genus Escherichia is M9 medium supplemented with glucose and Casamino acids (Miller, Journal of Experiments in Molecular Genetics, 431-433, Cold Spring Harbor Laboratory, New York, 1972). If necessary and desired, a chemical such as 3β-indolylacrylic acid can be added to the medium thereby to activate the promoter efficiently.

Where the bacteria belonging to the genus Escherichia are used as the host, the transformant is usually cultivated at about 15° C. to about 43° C. for about 3 hours to about 24 hours. If necessary and desired, the culture may be aerated or agitated.

Where the bacteria belonging to the genus Bacillus are used as the host, the transformant is cultivated generally at about 30° C. to about 40° C. for about 6 hours to about 24 hours. If necessary and desired, the culture can be aerated or agitated.

Where yeast is used as the host, the transformant is cultivated, for example, in Burkholder's minimal medium (Bostian, K. L. et al., Proc. Natl. Acad. Sci. U.S.A., 77, 4505 (1980)) or in SD medium supplemented with 0.5% Casamino acids (Bitter, G. A. et al., Proc. Natl. Acad. Sci. U.S.A., 81, 5330 (1984)). Preferably, pH of the medium is adjusted to about 5 to about 8. In general, the transformant is cultivated at about 20° C. to about 35° C. for about 24 hours to about 72 hours. If necessary and desired, the culture can be aerated or agitated.

Where insect cells or insects are used as the host, the transformant is cultivated in, for example, Grace's Insect Medium (Grace, T. C. C., Nature, 195, 788 (1962)) to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary and desired, the culture can be aerated or agitated.

Where animal cells are employed as the host, the transformant is cultivated in, for example, MEM medium containing about 5% to about 20% fetal bovine serum (Science, 122, 501 (1952)), DMEM medium (Virology, 8, 396 (1959)), RPMI 1640 medium (The Journal of the American Medical Association, 199, 519 (1967)), 199 medium (Proceeding of the Society for the Biological Medicine, 73, 1 (1950)), etc. Preferably, pH of the medium is adjusted to about 6 to about 8. The transformant is usually cultivated at about 30° C. to about 40° C. for about 15 hours to about 60 hours and, if necessary and desired, the culture can be aerated or agitated.

As described above, the protein of the present invention can be produced into the cell, in the cell membrane or out of the cell of the transformant.

The protein of the present invention can be separated and purified from the culture described above by the following procedures.

When the protein of the present invention is extracted from the culture or cells, after cultivation the transformants or cells are collected by a publicly known method and suspended in an appropriate buffer. The transformants or cells are then disrupted by publicly known methods such as ultrasonication, a treatment with lysozyme and/or freeze-thaw cycling, followed by centrifugation, filtration, etc. Thus, the crude extract of the protein of the present invention can be obtained. The buffer used for the procedures may contain a protein modifier such as urea or guanidine hydrochloride, or a surfactant such as Triton X-100™, etc. When the protein is secreted in the culture, after completion of the cultivation the supernatant can be separated from the transformants or cells to collect the supernatant by a publicly known method.

The receptor protein contained in the supernatant or the extract thus obtained can be purified by appropriately combining the publicly known methods for separation and purification. Such publicly known methods for separation and purification include a method utilizing difference in solubility such as salting out, solvent precipitation, etc.; a method utilizing mainly difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the protein thus obtained is in a free form, it can be converted into the salt by publicly known methods or modifications thereof. On the other hand, when the protein is obtained in the form of a salt, it can be converted into the free form or in the form of a different salt by publicly known methods or modifications thereof.

The protein produced by the recombinant can be treated, prior to or after the purification, with an appropriate protein modifying enzyme so that the receptor protein can be appropriately modified to partially remove a polypeptide. Examples of the protein-modifying enzyme include trypsin, chymotrypsin, arginyl endopeptidase, protein kinase, glycosidase or the like.

The activity of the thus produced protein of the present invention or salts thereof can be determined by a test binding to a labeled ligand, by an enzyme immunoassay using a specific antibody, or the like.

Antibodies to the protein of the present invention, its partial peptides, or salts thereof may be any of polyclonal antibodies and monoclonal antibodies, as long as they are capable of recognizing the receptor protein of the present invention, its partial peptides, or salts thereof.

The antibodies to the protein of the present invention, its partial peptides, or salts thereof (hereinafter sometimes merely referred to as the protein of the present invention) may be manufactured by publicly known methods for manufacturing antibodies or antisera, using as antigens the receptor protein of the present invention.

[Preparation of Monoclonal Antibody]

(a) Preparation of Monoclonal Antibody-Producing Cells

The protein of the present invention is administered to mammals either solely or together with carriers or diluents to the site where the production of antibody is possible by the administration. In order to potentiate the antibody productivity upon the administration, complete Freund's adjuvants or incomplete Freund's adjuvants may be administered. The administration is usually carried out once in every two to six weeks and 2 to 10 times in total. Examples of the applicable mammals are monkeys, rabbits, dogs, guinea pigs, mice, rats, sheep and goats, with mice and rats being preferred.

In the preparation of monoclonal antibody-producing cells, warm-blooded animals, e.g., mice, immunized with an antigen wherein the antibody titer is noted is selected, then the spleen or lymph node is collected after 2 to 5 days from the final immunization and antibody-producing cells contained therein are fused with myeloma cells to give monoclonal antibody-producing hybridomas. Measurement of the antibody titer in antisera may be made, for example, by reacting a labeled form of the protein of the present invention, which will be described later, with the antiserum followed by assaying the binding activity of the labeling agent bound to the antibody. The fusion may be operated, for example, by the known Koehler and Milstein method (Nature, 256, 495, 1975). Examples of the fusion accelerator are polyethylene glycol (PEG), Sendai virus, etc., of which PEG is preferably employed.

Examples of the myeloma cells are NS-1, P3U1, SP2/0, etc. In particular, P3U1 is preferably employed. A preferred ratio of the count of the antibody-producing cells used (spleen cells) to the count of myeloma cells is within a range of approximately 1:1 to 20:1. When PEG (preferably, PEG 1000 to PEG 6000) is added in a concentration of approximately 10 to 80% followed by incubating at about 20 to about 40° C., preferably at about 30 to about 37° C. for about 1 to about 10 minutes, an efficient cell fusion can be carried out.

Various methods can be used for screening of a monoclonal antibody-producing hybridoma. Examples of such methods include a method which comprises adding the supernatant of hybridoma to a solid phase (e.g., microplate) adsorbed with the protein etc. as an antigen directly or together with a carrier, adding an anti-immunoglobulin antibody (when mouse cells are used for the cell fusion, anti-mouse immunoglobulin antibody is used) labeled with a radioactive substance or an enzyme, or Protein A and detecting the monoclonal antibody bound to the solid phase, and a method which comprises adding the supernatant of hybridoma to a solid phase adsorbed with an anti-immunoglobulin antibody or Protein A, adding the protein labeled with a radioactive substance or an enzyme and detecting the monoclonal antibody bound to the solid phase.

The monoclonal antibody can be selected by publicly known methods or by modifications of these methods. In general, the selection can be effected in a medium for animal cells supplemented with HAT (hypoxanthine, aminopterin and thymidine). Any selection and growth medium can be employed as far as the hybridoma can grow therein. For example, RPMI 1640 medium containing 1% to 20%, preferably 10% to 20% fetal bovine serum, GIT medium (Wako Pure Chemical Industries, Ltd.) containing 1% to 10% fetal bovine serum, a serum free medium for cultivation of a hybridoma (SFM-101, Nissui Seiyaku Co., Ltd.) and the like can be used for the selection and growth medium. The cultivation is carried out generally at 20° C. to 40° C., preferably at about 37° C., for 5 days to 3 weeks, preferably 1 to 2 weeks. The cultivation can be conducted normally in 5% CO₂. The antibody titer of the culture supernatant of hybridomas can be determined as in the assay for the antibody titer in antisera described above.

(b) Purification of Monoclonal Antibody

Separation and purification of a monoclonal antibody can be carried out by methods applied to conventional separation and purification of immunoglobulins, as in the conventional methods for separation and purification of polyclonal antibodies [e.g., salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, Protein A, Protein G, etc. and dissociating the binding to obtain the antibody].

[Preparation of Polyclonal Antibody]

The polyclonal antibody of the present invention can be manufactured by publicly known methods or modifications thereof. For example, a complex of immunogen (an antigen such as the protein of the present invention) and a carrier protein is prepared, and a mammal is immunized with the complex in a manner similar to the method described above for the manufacture of monoclonal antibodies. The product containing the antibody to the protein of the present invention is collected from the immunized animal followed by separation and purification of the antibody.

In the complex of an immunogen and a carrier protein used to immunize a mammal, the type of carrier protein and the mixing ratio of a carrier to hapten may be any type and in any ratio, as long as the antibody is efficiently produced to the hapten immunized by crosslinking to the carrier. For example,. bovine serum albumin, bovine thyroglobulins, keyhole limpet hemocyanin, etc. is coupled to hapten in a carrier-to-hapten weight ratio of approximately 0.1 to 20, preferably about 1 to about 5.

A variety of condensing agents can be used for the coupling of a carrier to hapten. Glutaraldehyde, carbodiimide, maleimide-activated ester, activated ester reagents containing thiol group or dithiopyridyl group, etc. are used for the coupling.

The condensation product is administered to warm-blooded animals either solely or together with carriers or diluents to the site in which the antibody can be produce by the administration. In order to potentiate the antibody productivity upon the administration, complete Freund's adjuvant or incomplete Freund's adjuvant may be administered. The administration is usually made once approximately in every 2 to 6 weeks and about 3 to about 10 times in total.

The polyclonal antibody can be collected from the blood, ascites, etc., preferably from the blood of mammals immunized by the method described above.

The polyclonal antibody titer in antiserum can be assayed by the same procedure as that for the determination of serum antibody titer described above. The separation and purification of the polyclonal antibody can be carried out, following the method for the separation and purification of immunoglobulins performed as applied to the separation and purification of monoclonal antibodies described hereinabove.

The protein of the present invention, its partial peptide or their salts, and the DNA encoding them can be used for: (1) a method for determination of ligands to the protein of the present invention, (2) preparation of antibodies and antisera, (3) construction of recombinant protein expression systems, (4) development of the receptor binding assay systems using the expression systems and screening of pharmaceutical candidate compounds, (5) effecting drug design based on comparison with structurally similar ligand receptors, (6) reagents for preparation of probes and PCR primers for gene diagnosis, (7) preparation of transgenic animals, and (8) pharmaceutical for the gene prophylaxis and gene therapy.

In particular, by the use of the receptor binding assay system using the expression system of the recombinant protein of the present invention, compounds (e.g., agonists, antagonists, etc.) that alter the binding property of human- or mammal-specific ligands for the G protein-coupled receptor can be screened, and the agonists or antagonists can be used as prophylactic and therapeutic agents for various diseases.

Hereinafter, the protein of the present invention, its partial peptides, or salts thereof (hereinafter sometimes referred to as the protein of the present invention), the DNA encoding the protein of the present invention or its partial peptides (hereinafter sometimes referred to as the DNA of the present invention) and the antibodies to the protein of the present invention (hereinafter sometimes referred to as the antibodies of the present invention) are specifically described for the use or applications.

(1) A Method for Determination of a Ligand (Agonist, Antagonist) to the Protein of the Present Invention

The protein of the present invention or its salts, or the partial peptide or its salts of the present invention are useful as reagents for searching and determining ligands (agonists, antagonists) to the protein of the present invention or its salts.

That is, the present invention provides a method for determining a ligand to the protein of the present invention, which comprises bringing the protein of the present invention or its salts, or the partial peptide of the present invention or its salts, in contact with a test compound.

Examples of the test compound include publicly known ligands (e.g., angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotrienes, pancreastatin, prostaglandins, thromboxane, adenosine, adrenaline, α- and β-chemokines (e.g., IL-8, GROα, GROβ, GROγ, NAP-2, ENA-78, PF4, IP10, GCP-2, MCP-1, HC-14, MCP-3, I-309, MIP1α, MIP-1β, RANTES, etc.), endothelin, enterogastrin, histamine, neurotensin, TRH, pancreatic polypeptide, galanin, MIT1 or homologues derived from the mammal, etc.) as well as other substances, for example, tissue extracts and cell culture supernatants from human or mammals (e.g., mice, rats, swine, bovine, sheep, monkeys, etc.). Further, peptides containing an amino acid sequence represented by SEQ ID NO: 49, SEQ ID NO: 51, SEQ ID NO: 53, or SEQ ID NO: 71 can be used. For example, the tissue extract or cell culture supernatant is added to the protein of the present invention and fractionated while assaying the cell stimulating activities, etc. to finally give a single ligand.

Where the ligand is a peptide ligand, the ligand is sometimes referred to as a ligand peptide. In addition, where the ligand peptide is expressed as a precursor and changed to mature body by removing a signal peptide, each peptide is referred to as a ligand precursor peptide and a ligand mature peptide. However, both are collectively referred to as a ligand peptide.

In more detail, the method for determining ligands of the present invention comprises determining compounds (e.g., peptides, proteins, non-peptide compounds, synthetic compounds, fermentation products, etc.) or salts thereof that bind to the protein of the present invention to provide cell stimulating activities (e.g., the activities that accelerate or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), using the protein of the present invention, its partial peptides or salts thereof, or by the receptor binding assay using the recombinant protein expression system constructed.

The method for determining ligands of the present invention is characterized, for example, by measurement of the amount of the test compound bound to the protein or the partial peptide, or by assaying the cell-stimulating activities, etc., when the test compound is brought in contact with the protein of the present invention or its partial peptides.

More specifically, the present invention provides the following features:

\mathring{1} a method for determining a ligand to the protein of the present invention or its salt, which comprises bringing a labeled test compound in contact with the protein of the present invention or its salt or the partial peptide of the present invention or its salt and measuring the amount of the labeled test compound bound to the protein or its salt or to the partial peptide or its salt;

\mathring{2} a method for determining ligands to the protein of the present invention or its salt, which comprises bringing a labeled test compound in contact with cells or cell membrane fraction containing the protein of the present invention, and measuring the amount of the labeled test compound bound to the cells or the membrane fraction;

\mathring{3} a method for determining ligands to the protein of the present invention, which comprises culturing a transformant containing the DNA encoding the protein of the present invention, bringing a labeled test compound in contact with the protein expressed on the cell membrane by said culturing, and measuring the amount of the labeled test compound bound to the protein or its salt;

\mathring{4} a method for determining ligands to the protein of the present invention or its salt, which comprises bringing a test compound in contact with cells containing the protein of the present invention and measuring the protein-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.); and,

\mathring{5} a method for determining ligands to the protein of the present invention or its salt, which comprises culturing a transformant containing DNA encoding the protein of the present invention, bringing a labeled test compound in contact with the protein expressed on the cell membrane by said culturing, and measuring the protein-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.).

It is particularly preferred to perform the tests \mathring{1} to \mathring{2} described above, thereby to confirm that the test compound can bind to the protein of the present invention, followed by the tests \mathring{4} and \mathring{5} described above.

Any protein exemplified to be usable as the protein for determining ligands, so long as it contains the protein of the present invention or the partial peptide of the present invention. However, the protein that is abundantly expressed using animal cells is appropriate.

The protein of the present invention can be manufactured by the method for expression described above, preferably by expressing DNA encoding the protein in mammalian or insect cells. As DNA fragments encoding the desired portion of the protein, complementary DNA is generally used but not necessarily limited thereto. For example, gene fragments or synthetic DNA may also be used. For introducing a DNA fragment encoding the protein of the present invention into host animal cells and efficiently expressing the same, it is preferred to insert the DNA fragment downstream a polyhedrin promoter of nuclear polyhedrosis virus (NPV), which is a baculovirus having insect hosts, an SV40-derived promoter, a retrovirus promoter, a metallothionein promoter, a human heat shock promoter, a cytomegalovirus promoter, an SRα promoter or the like. The amount and quality of the receptor expressed can be determined by a publicly known method. For example, this determination can be made by the method described in the literature (Nambi, P., et al., J. Biol. Chem., 267, 19555-19559 (1992)).

Accordingly, the subject containing the protein of the present invention, its partial peptides or salts thereof in the method for determining the ligand according to the present invention may be the protein, its partial peptides or salts thereof purified by publicly known methods, cells containing the protein, or membrane fractions of such cells.

Where cells containing the protein of the present invention are used in the method of the present invention for determination of ligands, the cells may be fixed using glutaraldehyde, formalin, etc. The fixation can be made by a publicly known method.

The cells containing the protein of the present invention are host cells that have expressed the protein of the present invention, which host cells include Escherichia coli, Bacillus subtilis, yeast, insect cells, animal cells, and the like.

The cell membrane fraction refers to a fraction abundant in cell membrane obtained by cell disruption and subsequent fractionation by a publicly known method. Useful cell disruption methods include cell squashing using a Potter-Elvehjem homogenizer, disruption using a Waring blender or Polytron (manufactured by Kinematica Inc.), disruption by ultrasonication, and disruption by cell spraying through thin nozzles under an increased pressure using a French press or the like. Cell membrane fractionation is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the receptor protein expressed and membrane components such as cell-derived phospholipids and membrane proteins.

The amount of the protein in the cells containing the protein and in the membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To perform the methods \mathring{1} through \mathring{3} supra for determination of a ligand to the protein of the present invention or its salt, an appropriate protein fraction and a labeled test compound are required.

The protein fraction is preferably a fraction of naturally occurring receptor protein or a recombinant receptor fraction having an activity equivalent to that of the natural protein. Herein, the term “equivalent activity” is intended to mean a ligand binding activity, a signal transduction activity or the like that is equivalent to that possessed by naturally occurring receptor proteins.

Preferred examples of labeled test compounds include angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leukotrienes, pancreastatin, prostaglandins, thromboxane, adenosine, adrenaline, α- and β-chemokines (e.g., IL-8, GROα, GROβ, GROγ, NAP-2, ENA-78, PF4, IP10, GCP-2, MCP-1, HC-14, MCP-3, I-309, MIP1α, MIP-1β, RANTES, etc.), endothelin, enterogastrin, histamin, neurotensin, TRH, pancreatic polypeptide, galanin, MIT1 or homologues derived from the mammal, etc.), which are labeled with [³H], [¹²⁵I], [¹⁴C], [³⁵S], etc. Further, a peptide containing an amino acid sequence represented by SEQ ID NO: 49, SEQ ID NO: 51, SEQ ID NO: 53 or SEQ ID NO: 71 can be utilized.

More specifically, the ligand to the protein of the present invention or its salt is determined by the following procedures. First, a standard receptor preparation is prepared by suspending cells containing the protein of the present invention or the membrane fraction thereof in a buffer appropriate for use in the determination method. Any buffer can be used so long as it does not inhibit the binding between the ligand and the protein of the present invention, such buffers including a phosphate buffer or a Tris-HCl buffer having pH of 4 to 10 (preferably pH of 6 to 8). For the purpose of minimizing non-specific binding, a surfactant such as CHAPS, Tween-80™ (manufactured by Kao-Atlas Inc.), digitonin or deoxycholate, and various proteins such as bovine serum albumin or gelatin, may optionally be added to the buffer. Further for the purpose of suppressing the degradation of the receptors or ligands by proteases, a protease inhibitor such as PMSF, leupeptin, E-64 (manufactured by Peptide Institute, Inc.) and pepstatin may also be added. A given amount (5,000 to 500,000 cpm) of the test compound labeled with [³H], [¹²⁵I], [¹⁴C], [³⁵S] or the like is added to 0.01 ml to 10 ml of the receptor solution. To determine the amount of non-specific binding (NSB), a reaction tube containing an unlabeled test compound in large excess is also prepared. The reaction is carried out at approximately 0 to 50° C., preferably about 4 to 37° C. for about 20 minutes to about 24 hours, preferably about 30 minutes to about 3 hours. After completion of the reaction, the reaction mixture is filtrated through glass fiber filter paper, etc. and washed with an appropriate volume of the same buffer. The residual radioactivity on the glass fiber filter paper is then measured by means of a liquid scintillation counter or γ-counter. A test compound exceeding 0 cpm in count obtained by subtracting nonspecific binding (NSB) from the total binding (B) (B minus NSB) may be selected as a ligand (agonist) to the protein of the present invention or its salt.

The method \mathring{4} or \mathring{5} above for determination of a ligand to the protein of the present invention or its salt can be performed as follows. The protein-mediated cell-stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) may be determined by a publicly known method, or using an assay kit commercially available. Specifically, cells containing the protein of the present invention are first cultured on a multi-well plate, etc. Prior to the ligand determination, the medium is replaced with fresh medium or with an appropriate non-cytotoxic buffer, followed by incubation for a given period of time in the presence of a test compound, etc. Subsequently, the cells are extracted or the supernatant is recovered and the resulting product is quantified by appropriate procedures. Where it is difficult to detect the production of the index substance (e.g., arachidonic acid) for the cell-stimulating activity due to a degrading enzyme contained in the cells, an inhibitor against such a degrading enzyme may be added prior to the assay. For detecting activities such as the cAMP production suppression activity, the baseline production in the cells is increased by forskolin or the like and the suppressing effect on the increased baseline production may then be detected.

The kit of the present invention for determination of the ligand that binds to the protein of the present invention or its salt comprises the protein of the present invention or its salt, the partial peptide of the present invention or its salt, cells containing the protein of the present invention, or the membrane fraction of the cells containing the protein of the present invention.

Examples of the ligand determination kit of the present invention are given below.

1. Reagents for Determining Ligands

\mathring{1} Buffers for Assay and Washing

Hanks' Balanced Salt Solution (manufactured by Gibco Co.) supplemented with 0.05% bovine serum albumin (Sigma Co.).

The solution is sterilized by filtration through a 0.45 μm filter and stored at 4° C. Alternatively, the solution may be prepared at use.

\mathring{2} Standard G Protein-Coupled Receptor Protein

CHO cells on which the protein of the present invention has been expressed are passaged in a 12-well plate in a density of 5×10⁵ cells/well followed by culturing at 37° C. under 5% CO₂ and 95% air for 2 days.

\mathring{3} Labeled Test Compounds

Compounds labeled with [³H], [¹²⁵I], [¹⁴C], [³⁵S], etc., which are commercially available labels, or compounds labeled by appropriate methods.

An aqueous solution of the compound is stored at 4° C. or −20° C. The solution is diluted to 1 μM with an assay buffer at use. A sparingly water-soluble test compound is dissolved in dimethylformamide, DMSO, methanol, etc.

\mathring{4} Non-Labeled Compounds

A non-labeled form of the same compound as the labeled compound is prepared in a concentration 100 to 1,000-fold higher than that of the labeled compound.

2. Method for Assay

\mathring{1} CHO cells expressing the protein of the present invention are cultured in a 12-well culture plate. After washing twice with 1 ml of an assay buffer, 490 μl of the assay buffer is added to each well.

\mathring{2} After 5 μl of the labeled test compound is added, the resulting mixture is incubated at room temperature for an hour. To determine the non-specific binding, 5 μl of the non-labeled compound is added to the system.

\mathring{3} The reaction mixture is removed and the wells are washed 3 times with 1 ml of washing buffer. The labeled test compound bound to the cells is dissolved in 0.2N NaOH-1% SDS and then mixed with 4 ml of liquid scintillator A (manufactured by Wako Pure Chemical Industries, Ltd.).

\mathring{4} The radioactivity is measured using a liquid scintillation counter (manufactured by Beckman Co.).

The ligands that are able to bind to the protein of the present invention or its salt include substances specifically present in the brain, pituitary gland, heart, pancreas, and the like. Specific examples of such ligands are angiotensin, bombesin, canavinoid, cholecystokinin, glutamine, serotonin, melatonin, neuropeptide Y, opioids, purines, vasopressin, oxytocin, PACAP, secretin, glucagon, calcitonin, adrenomedulin, somatostatin, GHRH, CRF, ACTH, GRP, PTH, VIP (vasoactive intestinal-peptide), somatostatin, dopamine, motilin, amylin, bradykinin, CGRP (calcitonin gene-related peptide), leukotriens, pancreastatin, prostaglandins, thromboxane, adenosine, adrenaline, α- and β-chemokines (e.g., IL-8, GROα, GROβ, GROγ, NAP-2, ENA-78, PF4, IP10, GCP-2, MCP-1, HC14, MCP-3, I-309, MIP1α, MIP-1β, RANTES, etc.), endothelin, enterogastrin, histamine, neurotensin, TRH, pancreatic polypeptide, galanin, MIT1 or homologues derived from the mammal, etc.

(2) Prophylactic and/or Therapeutic Agents for Deficiency in the Protein of the Present Invention

When a compound is clarified to be a ligand of the protein of the present invention by the methods described in (1), \mathring{1} the protein of the present invention, or \mathring{2} the DNA encoding the protein can be used, depending on the activities possessed by the ligand, as a prophylactic and/or therapeutic agent for diseases associated with dysfunction of the protein of the present invention.

For example, when the physiological activity of the ligand cannot be expected in a patient (deficiency of the protein) due to a decrease in the protein of the present invention in vivo, the activity of the ligand can be exhibited by: \mathring{1} administering the protein of the present invention to the patient thereby to supplement the amount of the protein; or \mathring{2} by increasing the amount of the protein in the patient through: i) administration of the DNA encoding the protein of the present invention to express the same in the patient; or ii) insertion and expression of the DNA encoding the protein of the present invention in the objective cells to transplant the cells to the patient, whereby the activity of the ligand can be sufficiently exhibited. Thus, the DNA encoding the protein of the present invention is useful as a safe and low toxic pharmaceutical such as a prophylactic and/or therapeutic agent for diseases associated with dysfunction of the receptor protein of the present invention.

The protein or the DNA encoding the protein of the present invention are useful for the prevention and/or treatment of central dysfunction (e.g., Alzheimer's disease, senile dementia, suppression of eating (cibophobia), epilepsia, etc.), diseases associated with hormone system (e.g., uterine inertia, atonic bleeding, placental dystocia, incomplete involution of the uterus, caesarean section, artificial abortion, retention of milk, etc.), diseases associated with liver, cholecyst, pancreas and incretion (e.g., diabetes, suppression of eating, etc.), inflammatory diseases (e.g., allergy, asthma, rheumatoid, etc.), circulatory diseases (e.g., hypertension, cardiac hypertrophy, angina pectoris, arteriosclerosis, etc.), respiratory diseases (e.g., pneumonia, asthma, bronchitis, respiratory infection, chronic obstructive pulmonary disease, etc.), infectious diseases (e.g., sepsis, MRSA, respiratory infection, urinary tract infection, biliary tract infection, infectious enteritis, inflammation of middle ear, prostatitis, etc.).

In particular, the protein of the present invention or the DNA encoding the protein is useful in prevention and/or treatment of alimentary diseases (e.g., enteritis, diarrheal disease, constipation, malabsorption syndrome, etc.).

When the protein of the present invention is used as the prophylactic/therapeutic agents supra, the protein can be prepared into a drug product in a conventional manner.

On the other hand, where the DNA encoding the protein of the present invention (hereinafter sometimes referred to as the DNA of the present invention) is used as the prophylactic/therapeutic agents described above, the DNA itself is administered; alternatively, the DNA is inserted into an appropriate vector such as retrovirus vector, adenovirus vector, adenovirus-associated virus vector, etc. and then administered in a conventional manner. The DNA of the present invention may also be administered as naked DNA, or with adjuvants to assist its uptake by gene gun or through a catheter such as a catheter with a hydrogel.

For example, \mathring{1} the protein of the present invention or \mathring{2} the DNA encoding the protein can be used orally, for example, in the form of tablets which may be sugar coated if necessary and desired, capsules, elixirs, microcapsules etc., or parenterally in the form of injectable preparations such as a sterile solution and a suspension in water or with other pharmaceutically acceptable liquid. These preparations can be manufactured by mixing \mathring{1} the protein of the present invention or \mathring{2}the DNA encoding the protein with a physiologically acceptable known carrier, a flavoring agent, an excipient, a vehicle, an antiseptic agent, a stabilizer, a binder, etc. in a unit dosage form required in a generally accepted manner that is applied to making pharmaceutical preparations. The effective component in the preparation is controlled in such a dose that an appropriate dose is obtained within the specified range given.

Additives miscible with tablets, capsules, etc. include a binder such as gelatin, corn starch, tragacanth and gum arabic, an excipient such as crystalline cellulose, a swelling agent such as corn starch, gelatin and alginic acid, a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated by conventional procedures used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition. Examples of an aqueous medium for injection include physiological saline and an isotonic solution containing glucose and other auxiliary agents (e.g., D-sorbitol, D-mannitol, sodium chloride, etc.) and may be used in combination with an appropriate dissolution aid such as an alcohol (e.g., ethanol or the like), a polyalcohol (e.g., propylene glycol and polyethylene glycol), a nonionic surfactant (e.g., polysorbate 80™ and HCO-50), etc. Examples of the oily medium include sesame oil and soybean oil, which may also be used in combination with a dissolution aid such as benzyl benzoate and benzyl alcohol.

The prophylactic/therapeutic agent described above may further be formulated with a buffer (e.g., phosphate buffer, sodium acetate buffer, etc.), a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus-prepared liquid for injection is normally filled in an appropriate ampoule.

Since the thus obtained pharmaceutical preparation is safe and low toxic, the preparation can be administered to human or mammal (e.g., rats, rabbits, sheep, swine, bovine, cats, dogs, monkeys, etc.).

The dose of the protein or the DNA of the present invention varies depending on subject to be administered, organs to be administered, conditions, routes for administration, etc.; in oral administration, e.g., for the adult patient with alimentary diseases, the dose is normally about 0.1 mg to about 100 mg, preferably about 1.0 to about 50 mg, and more preferably about 1.0 to about 20 mg per day (as 60 kg body weight). In parenteral administration, the single dose varies depending on subject to be administered, target organ, conditions, routes for administration, etc. but it is advantageous, e.g., for the adult patient with alimentary diseases, to administer the active ingredient intravenously in a daily dose of about 0.01 to about 30 mg, preferably about 0.1 to about 20 mg, and more preferably about 0.1 to about 10 mg (as 60 kg body weight). For other animal species, the corresponding dose as converted per 60 kg body weight can be administered.

(3) Gene Diagnostic Agent

By using the DNA of the present invention as a probe, an abnormality (gene abnormality) of the DNA or mRNA encoding the protein of the present invention or its partial peptide in human or mammal (e.g., rats, rabbits, sheep, swine, bovine, cats, dogs, monkeys, etc.) can be detected. Therefore, the DNA of the present invention is useful as a gene diagnostic agent for the damage of the DNA or mRNA, its mutation, or its decreased expression, or increased expression or overexpression of the DNA or mRNA.

The gene diagnosis described above using the DNA of the present invention can be performed by, for example, the publicly known Northern hybridization assay or the PCR-SSCP assay (Genomics, 5, 874-879 (1989); Proceedings of the National Academy of Sciences of the United States of America, 86, 2766-2770 (1989)).

(4) Methods of Quantifying Ligands for the Protein of the Present Invention

Since the protein etc. of the present invention has binding affinity to ligands, concentration of the ligand can be quantified in vivo with good sensitivity.

The quantification methods of the present invention can be used in combination with, for example, a competitive method. The concentration of the ligand in a test sample can be measured by contacting the test sample to the protein etc. of the present invention. Specifically, the methods can be used by following, for example, the methods described in \mathring{1} and \mathring{2} below or its modified methods.

\mathring{1} Hiroshi Irie, ed. “Radioimmunoassay,” Kodansha, published in 1974

\mathring{2} Hiroshi Irie, ed. “Sequel to the Radioimmunoassay,” Kodansha, published in 1979

(5) Methods of Screening Compounds that Alter the Binding Property Between the Protein of the Present Invention and Ligands

Using the protein etc. of the present invention, or constructing the expression system of the recombinant protein etc. to use the receptor binding assay system, compounds (e.g., peptides, proteins, non-peptide compounds, synthetic compounds, fermentation products, etc.) or salts thereof that alter the binding property between ligands and the protein of the present invention can be efficiently screened.

Such compounds include (a) compounds that have the G protein-coupled receptor-mediated cell-stimulating activities (e.g., activities that promote or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.) (so-called agonists to the protein of the present invention); (b) compounds that do not have the cell-stimulating activity (so-called antagonists to the protein of the present invention); (c) compounds that potentiate the binding affinity between ligands and the protein of the present invention; and (d) compounds that reduce the binding affinity between ligands and the protein of the present invention (it is preferred to screen the compounds described in (a) using the ligand determination methods described above).

That is, the present invention provides methods of screening compounds or their salts that alter the binding property between ligands and the protein, its partial peptide or salts thereof, which comprises comparing (i) the case wherein the protein of the present invention, its partial peptide or salts thereof are brought in contact with a ligand, with (ii) the case wherein the protein of the present invention, its partial peptide or salts thereof are brought in contact with a ligand and a test compound.

The screening methods of the present invention are characterized by assaying, for example, the amount of ligand bound to the protein etc., the cell-stimulating activity, etc., and comparing the property between (i) and (ii).

More specifically, the present invention provides the following screening methods:

a) A method of screening a compound or its salt that alters the binding property between a ligand and the protein etc. of the present invention, which comprises:

measuring the amount of a labeled ligand bound to the protein etc., when the labeled ligand is brought in contact with the protein etc. of the present invention and when the labeled ligand and a test compound are brought in contact with the protein etc. of the present invention, and,

comparing the binding property between them;

b) A method of screening a compound or its salt that alters the binding property between a ligand and the protein etc. of the present invention, which comprises:

measuring the amount of a labeled ligand bound to cells or the membrane fraction of the cells, when the labeled ligand is brought in contact with the cells or cell membrane fraction containing the protein etc. of the present invention and when the labeled ligand and a test compound are brought in contact with the cells or cell membrane fraction containing the protein etc. of the present invention, and,

comparing the binding property between them;

c) A method of screening a compound or its salt that alters the binding property between a ligand and the protein etc. of the present invention, which comprises:

measuring the amount of a labeled ligand to the protein etc., when the labeled ligand is brought in contact with the protein etc. expressed on the cell membrane induced by culturing a transformant containing the DNA of the present invention and when the labeled ligand and a test compound are brought in contact with the protein etc. of the present invention expressed on the cell membrane induced by culturing a transformant containing the DNA of the present invention, and,

comparing the binding property between them;

d) A method of screening a compound or its salt that alters the binding property between a ligand and the protein etc. of the present invention, which comprises:

measuring the receptor-mediated cell-stimulating activity (e.g., the activity that promotes or suppresses arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.), when a compound (e.g., a ligand to the protein etc. of the present invention) that activates the protein etc. of the present invention is brought in contact with cells containing the protein etc. of the present invention and when the compound that activates the protein etc. of the present invention and a test compound are brought in contact with cells containing the protein etc. of the present invention, and,

comparing the binding property between them; and,

e) A method of screening a compound or its salt that alters the binding property between a ligand and the protein etc. of the present invention, which comprises:

measuring the receptor-mediated cell-stimulating activity (e.g., the activity that promotes or suppresses arachidonic acid release, acetylcholine release, intracellular Ca²⁺ release, intracellular CAMP production, intracellular cGMP production, inositol phosphate production, changes in cell membrane potential, phosphorylation of intracellular proteins, activation of c-fos, pH reduction, etc.), when a compound (e.g., a ligand for the protein etc. of the present invention) that activates the protein etc. of the present invention is brought in contact with the protein etc. of the present invention expressed on the cell membrane induced by culturing a transformant containing the DNA of the present invention and when the compound that activates the protein etc. of the present invention and a test compound are brought in contact with the protein etc. of the present invention expressed on the cell membrane induced by culturing a transformant containing the DNA of the present invention, and,

comparing the binding property between them.

Before the protein etc. of the present invention was obtained, it was required for screening G protein-coupled receptor agonists or antagonists to obtain candidate compounds first, using cells or tissues containing the G protein-coupled receptor protein or the cell membrane fraction from rats or other animals (primary screening), and then examine the candidate compounds whether the compounds actually inhibit the binding between human G protein-coupled receptor protein and ligands (secondary screening). When cells, tissues, or the cell membrane fractions were directly used, it was practically difficult to screen agonists or antagonists to the objective receptor protein, since other receptor proteins were present together.

However, using, for example, the rat-derived protein of the present invention, the primary screening becomes unnecessary, and compounds that inhibit the binding between ligands and the G protein-coupled receptor protein can be efficiently screened. Furthermore, it is easy to assess whether the obtained compound is an agonist or antagonist.

Hereinafter, the screening methods of the present invention are described more specifically.

First, for the protein etc. of the present invention used for the screening methods of the present invention, any substance may be used so long as it contains the protein etc. of the present invention described above. The cell membrane fraction from mammalian organs containing the protein etc. of the present invention is preferred. However, it is very difficult to obtain human organs. It is thus preferable to use rat-derived receptor proteins or the like, produced by large-scale expression using recombinants.

To manufacture the protein etc. of the present invention, the methods described above are used, and it is preferred to express the DNA of the present invention in mammalian and insect cells. For the DNA fragment encoding the objective protein region, the complementary DNA, but not necessarily limited thereto, is employed. For example, the gene fragments and synthetic DNA may also be used. To introduce a DNA fragment encoding the protein of the present invention into host animal cells and efficiently express the DNA there, it is preferred to insert the DNA fragment downstream of a polyhedorin promoter of nuclear polyhedrosis virus (NPV) belonging to baculovirus hosted by insects, SV40-derived promoter, retrovirus promoter, metallothionein promoter, human heat shock promoter, cytomegalovirus promoter, or SRα promoter. The amount and quality of the expressed receptor are examined by publicly known methods, for example, the method described in the literature [Nambi, P. et al., The Journal of Biological Chemistry (J. Biol. Chem.), 267, 19555-19559, 1992].

Therefore, in the screening methods of the present invention, the material that contains the protein etc. of the present invention may be the protein etc. purified by publicly known methods, cells containing the protein etc., or the cell membrane fraction containing the protein or the like.

In the screening methods of the present invention, when cells containing the protein etc. of the present invention are used, the cells may be fixed with glutaraldehyde, formalin, etc. The cells can be fixed by publicly known methods.

The cells containing the protein etc. of the present invention are host cells that have expressed the protein or the like. For the host cells, Escherichia coli, Bacillus subtilis, yeast, insect cells, animal cells and the like are preferred.

The cell membrane fraction refers to a fraction abundant in cell membrane obtained by cell disruption and subsequent fractionation by a publicly known method. Useful cell disruption methods include cell squashing using a Potter-Elvehjem homogenizer, disruption using a Waring blender or Polytron (manufactured by Kinematica Inc.), disruption by ultrasonication, and disruption by cell spraying through thin nozzles under an increased pressure using a French press or the like. Cell membrane fractionation is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the protein etc. expressed and membrane components such as cell-derived phospholipids and membrane proteins.

The amount of the protein in the cells containing the protein etc. and in the membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To screen the compounds that alter the binding property between ligands and the protein etc. of the present invention described in a) to c), for example, an appropriate protein fraction and a labeled ligand are necessary.

The protein fraction is preferably a fraction of naturally occurring receptor protein or a recombinant receptor protein fraction having an activity equivalent to that of the natural protein. Herein, the equivalent activity is intended to mean a ligand binding activity, a signal transduction activity or the like that is equivalent to that possessed by naturally occurring receptor proteins.

For the labeled ligand, a labeled ligand and a labeled ligand analogue are used. For example, ligands labeled with [³H], [¹²⁵I], [¹⁴C], [³⁵S], etc. are used.

Specifically, to screen the compounds that alter the binding property between ligands and the protein etc. of the present invention, first, the protein standard is prepared by suspending cells or cell membrane fraction containing the protein etc. of the present invention in a buffer appropriate for the screening. For the buffer, any buffer that does not interfere with the binding of ligands to the protein is usable and examples of such a buffer are phosphate buffer, Tris-hydrochloride buffer, etc., having pH of 4 to 10 (preferably pH of 6 to 8). To minimize a non-specific birding, a surfactant such as CHAPS, Tween-80™ (Kao-Atlas Co.), digitonin, deoxycholate, etc. may be added to the buffer. To inhibit degradation of the receptor and ligands by proteases, protease inhibitors such as PMSF, leupeptin, E-64 (manufactured by Peptide Research Laboratory, Co.), and pepstatin may be added. To 0.01 to 10 ml of the receptor solution, a given amount (5,000 to 500,000 cpm) of labeled ligand is added, and 10⁻⁴ M-10⁻¹⁰ M of a test compound is simultaneously added to be co-present. To examine non-specific binding (NSB), a reaction tube containing an unlabeled test compound in large excess is also prepared. The reaction is carried out at approximately 0 to 50° C., preferably about 4 to 37° C. for about 20 minutes to about 24 hours, preferably about 30 minutes to about 3 hours. After completion of the reaction, the reaction mixture is filtrated through glass fiber filter paper, etc. and washed with an appropriate volume of the same buffer. The residual radioactivity on the glass fiber filter paper is then measured by means of a liquid scintillation counter or γ-counter. Regarding the count obtained by subtracting the amount of non-specific binding (NSB) from the count obtained in the absence of any competitive substance (B₀) as 100%, when the amount of specific binding (B-NSB) is, for example, 50% or less, the test compound can be selected as a candidate substance having a potential of competitive inhibition.

To perform the methods d) and e) supra of screening the compounds that alter the binding property between ligands and the protein etc. of the present invention, the protein-mediated cell-stimulating activity (e.g., activity that promotes or inhibits arachidonic acid release, acetylcholine release, intracellular Ca release, intracellular cAMP production, intracellular cGMP production, inositol phosphate production, changes in cell membrane potential, phosphorylation of intracellular proteins, activation of c-fos, pH reduction, etc.) can be measured using publicly known methods or commercially available kits.

Specifically, the cells containing the protein etc. of the present invention are first cultured on a multi-well plate, etc. Prior to screening, the medium is replaced with fresh medium or with an appropriate non-cytotoxic buffer, followed by incubation for a given period of time in the presence of a test compound, etc. Subsequently, the cells are extracted or the supernatant is recovered and the resulting product is quantified by appropriate procedures. Where it is difficult to detect the production of the index substance (e.g., arachidonic acid) for the cell-stimulating activity due to a degrading enzyme contained in the cells, an inhibitor against such a degrading enzyme may be added prior to the assay. For detecting activities such as the cAMP production suppression activity, the baseline production in the cells is increased by forskolin or the like and the suppressing effect on the increased baseline production may then be detected.

Screening by assaying the cell-stimulating activity requires cells that have expressed an appropriate protein. For the cells that have expressed the protein etc. of the present invention, the cell line possessing the native protein etc. of the present invention, the cell line expressing the recombinant protein described above and the like are desirable.

For the test compound, for example, peptides, proteins, non-peptide compounds, synthetic compounds, fermentation products, cell extracts, plant extracts, and animal tissue extracts are used. These compounds may be novel or known compounds.

The kits for screening the compounds or their salts that alter the binding property between ligands and the protein etc. of the present invention comprise the protein etc. of the present invention, cells containing the protein etc. of the present invention, or the membrane fraction of cells containing the protein etc. of the present invention.

Examples of the screening kits of the present invention are as follow.

1. Reagents for Screening

\mathring{1} Buffer for Measurement and Washing

Hanks' balanced salt solution (manufactured by. Gibco Co.) supplemented with 0.05% bovine serum albumin (manufactured by Sigma Co.).

The solution is sterilized by filtration through a 0.45 μm filter, and stored at 4° C. or may be prepared at use.

\mathring{2} Standard G Protein-Coupled Receptor

CHO cells expressing the protein of the present invention are passaged in a 12-well plate at a density of 5×10⁵ cells/well followed by culturing at 37° C. under 5% CO₂ and 95% air for 2 days.

\mathring{3} Labeled Ligands

Ligands labeled with commercially available [³H], [¹²⁵I], [¹⁴C], [³⁵S]etc.

The aqueous solutions are stored at 4° C. or −20° C., and diluted to 1 μM with the measurement buffer.

\mathring{4} Standard Ligand Solution

The ligand is dissolved in and adjusted to 1 mM with PBS containing 0.1% bovine serum albumin (manufactured by Sigma Co.) and stored at −20° C.

2. Measurement Method

\mathring{1} CHO cells expressing the protein of the present invention are cultured in a 12-well culture plate and washed twice with 1 ml of the measurement buffer, and 490 μl of the measurement buffer is added to each well.

\mathring{2} After adding 5 μl of 10⁻³-10⁻¹⁰ M test compound solution, 5 μl of a labeled ligand is added to the mixture, and the cells are incubated at room temperature for an hour. To determine the amount of the non-specific binding, 5 μl of the non-labeled ligand is added in place of the test compound.

\mathring{3} The reaction solution is removed, and the wells are washed 3 times with the washing buffer. The labeled ligand bound to the cells is dissolved in 0.2N NaOH-1% SDS, and mixed with 4 ml of liquid scintillator A (manufactured by Wako Pure Chemical Industries, Ltd.)

\mathring{4} The radioactivity is measured using a liquid scintillation counter (manufactured by Beckman Co.), and the percent maximum binding (PMB) is calculated by the equation below.

PMB=[(B−NSB)/(B₀−NSB)]×100

PMB: Percent maximum binding

B: Value obtained in the presence of a test compound

NSB: Non-specific binding

B₀: Maximum binding

The compounds or their salts obtained using the screening methods or the screening kits of the present invention are the compounds that alter the binding property between ligands and the protein etc. of the present invention. Specifically, these compounds are: (a) compounds having the G protein-coupled receptor-mediated cell-stimulating activity (e.g., activity that promotes or inhibits arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.) (so-called agonists to the protein of the present invention); (b) compounds having no cell stimulating-activity (so-called antagonists to the protein of the present invention); (c) compounds that potentiate the binding affinity between ligands and the G protein-coupled protein of the present invention; and (d) compounds that reduce the binding affinity between ligands and the G protein-coupled protein of the present invention.

The compounds may be peptides, proteins, non-peptide compounds, synthetic compounds, fermentation products, and may be novel or known compounds.

Since agonists to the protein etc. of the present invention have the same physiological activities as those of the ligands for the protein etc. of the present invention, the agonists are useful as safe and low toxic pharmaceuticals (e.g., a prophylactic and/or therapeutic agent for central dysfunction (e.g., Alzheimer's disease, senile dementia, suppression of eating (cibophobia), epilepsia, etc.), diseases associated with hormone system (e.g., uterine inertia, atonic bleeding, placental dystocia, incomplete involution of the uterus, caesarean section, artificial abortion, retention of milk, etc.), diseases associated with liver, cholecyst, pancreas and incretion (e.g., diabetes, suppression of eating, etc.), inflammatory diseases (e.g., allergy, asthma, rheumatoid, etc.), circulatory diseases (e.g., hypertension, cardiac hypertrophy, angina pectoris, arteriosclerosis, etc.), respiratory diseases (e.g., pneumonia, asthma, bronchitis, respiratory infection, chronic obstructive pulmonary disease, etc.), infectious diseases (e.g., sepsis, MRSA, respiratory infection, urinary tract infection, biliary tract infection, infectious enteritis, inflammation of middle ear, prostatitis, etc.), etc.), correspondingly to the ligand activities.

In particular, the agonists to the protein of the present invention are useful as a safe and low toxic prophylactic and/or therapeutic agent for alimentary diseases (e.g., enteritis, diarrheal disease, constipation, malabsorption syndrome, etc.) depending on the ligand activities, since they have the same physiological activities as those of the ligands for the protein etc. of the present invention.

Since antagonists to the protein etc. of the present invention can suppress the physiological activities of ligands for the protein etc. of the present invention, the antagonists are useful as safe and low toxic pharmaceuticals that inhibit the ligand activities (e.g., a regulant of hormone secretion, a prophylactic and/or therapeutic agent for central nerve diseases raised by excess production of the ligand to the protein of the present invention, diseases associated with hormone system, diseases associated with liver, cholecyst, pancreas and incretion (e.g., anti-obesity drugs, excess of eating, etc.), inflammatory diseases, circulatory diseases, respiratory diseases, infectious diseases, etc.).

Since antagonists to the protein etc. of the present invention can suppress the physiological activities of ligands for the protein etc. of the present invention, they are particularly useful as a safe and low toxic prophylactic and/or therapeutic agent for alimentary diseases (e.g., enteritis, diarrheal disease, constipation, malabsorption syndrome, etc.) that suppress the ligand activities. The compounds that reduce the binding affinity between ligands and the protein of the present invention are useful as safe and low toxic pharmaceuticals that decrease the physiological activities of ligands for the protein etc. of the present invention (e.g., a regulant of hormone secretion, a prophylactic and/or therapeutic agent for central nerve diseases raised by excess production of the ligand to the protein of the present invention, diseases associated with hormone system, diseases associated with liver, cholecyst, pancreas and incretion (e.g., anti-obesity drugs, excess of eating, etc.), inflammatory diseases, circulatory diseases, respiratory diseases, infectious diseases, etc.).

Since the compounds that reduce the binding affinity between ligands and the protein of the present invention can reduce the physiological activities, which the ligand to the protein of the present invention has, they are particularly useful as a safe and low toxic prophylactic and/or therapeutic agent for alimentary diseases (e.g., enteritis, diarrheal disease, constipation, malabsorption syndrome, etc.).

When compounds or their salts, which are obtained by the screening methods or using the screening kits of the present invention, are employed as the pharmaceutical compositions described above, the compounds can be formulated in the pharmaceutical preparations in a conventional manner. For example, the compounds can be prepared into tablets, capsules, elixir, microcapsules, aseptic solution, suspension, etc., as described for pharmaceuticals containing the protein of the present invention.

The preparations thus obtained are safe and low toxic, and can be administered to, for example, human and mammals (e.g., rats, rabbits, sheep, swine, bovine, cats, dogs, monkeys, etc.).

The dose of the compounds or their salt forms varies depending on subject to be administered, target organs, conditions, routes for administration, etc.; in oral administration, e.g., for the adult, the dose is normally about 0.1 mg to about 100 mg, preferably about 1.0 to about 50 mg, and more preferably about 1.0 to about 20 mg per day (as 60 kg body weight). In parenteral administration, the single dose varies depending on subject to be administered, target organ, conditions, routes for administration, etc. but it is advantageous, e.g., for the adult patient with alimentary diseases, to administer the active ingredient intravenously in a daily dose of about 0.01 to about 30 mg, preferably about 0.1 to about 20 mg, and more preferably about 0.1 to about 10 mg (as 60 kg body weight). For other animal species, the corresponding dose as converted per 60 kg body weight can be administered.

(6) Quantification of the Protein of the Present Invention, Its Partial Peptide, or Its Salt

The antibodies of the present invention are capable of specifically recognizing the protein etc. of the present invention. Therefore, the antibodies can be used to quantify the protein etc. of the present invention in a test fluid, especially for quantification by the sandwich immunoassay. That is, the present invention provides, for example, the following quantification methods:

(i) A method of quantifying the protein etc. of the present invention in a test fluid, which comprises competitively reacting the antibody of the present invention with the test fluid and a labeled form of the protein etc. of the present invention, and measuring the ratio of the labeled protein etc. bound to the antibody; and,

(ii) A method of quantifying the protein etc. of the present invention in a test fluid, which comprises reacting the test fluid with the antibody of the present invention immobilized on a carrier and a labeled form of the antibody of the present invention simultaneously or sequentially, and measuring the activity of the label on the immobilized carrier.

In (ii) described above, it is preferred that one antibody recognizes the N-terminal region of the protein etc. of the present invention, and another antibody reacts with the C-terminal region of the protein etc. of the present invention.

Using monoclonal antibodies to the protein etc. of the present invention (hereinafter sometimes referred to as the monoclonal antibodies of the present invention), the protein etc. of the present invention can be assayed and also detected by tissue staining or the like. For this purpose, an antibody molecule itself may be used, or F(ab′)₂, Fab′ or Fab fractions of the antibody molecule may also be used. Assay methods using antibodies to the protein etc. of the present invention are not particularly limited. Any assay method can be used, so long as the amount of antibody, antigen, or antibody-antigen complex corresponding to the amount of antigen (e.g., the amount of the protein) in the test fluid can be detected by chemical or physical means and the amount of the antigen can be calculated from a standard curve prepared from standard solutions containing known amounts of the antigen. For example, nephrometry, competitive methods, immunometric method, and sandwich method are appropriately used, with the sandwich method described below being most preferable in terms of sensitivity and specificity.

As the labeling agent for the methods using labeled substances, there are employed, for example, radioisotopes, enzymes, fluorescent substances, luminescent substances, etc. For the radioisotope, for example, [¹²⁵I], [¹³¹ ], [³H] and [¹⁴C] are used. As the enzyme described above, stable enzymes with high specific activity are preferred; for example, β-galactosidase, β-glucosidase, alkaline phosphatase, peroxidase, malate dehydrogenase and the like are used. Example of the fluorescent substance used is fluorescamine and fluorescein isothiocyanate are used. For the luminescent substance, for example, luminol, luminol derivatives, luciferin, and lucigenin are used. Furthermore, the biotin-avidin system may be used for binding antibody or antigen to the label.

For immobilization of antigen or antibody, physical adsorption may be used. Chemical binding methods conventionally used for insolubilization or immobilization of proteins or enzymes may also be used. For the carrier, for example, insoluble polysaccharides such as agarose, dextran, cellulose, etc.; synthetic resin such as polystyrene, polyacrylamide, silicon, etc., and glass or the like are used.

In the sandwich method, the immobilized monoclonal antibody of the present invention is reacted with a test fluid (primary reaction), then with the labeled monoclonal antibody of the present invention (secondary reaction), and the activity of the label on the immobilizing carrier is measured, whereby the amount of the protein of the present invention in the test fluid can be quantified. The order of the primary and secondary reactions may be reversed, and the reactions may be performed simultaneously or with an interval. The labeling agent and the methods of immobilization can be performed by the methods described above.

In the immunoassay by the sandwich method, the antibody used for immobilized or labeled antibodies is not necessarily one species, but a mixture of two or more species of antibody may be used to increase the measurement sensitivity.

In the methods of assaying the protein etc. of the present invention by the sandwich method, antibodies that bind to different sites of the protein etc. are preferably used as the monoclonal antibodies of the present invention for the primary and secondary reactions. That is, in the antibodies used for the primary and secondary reactions, for example, when the antibody used in the secondary reaction recognizes the C-terminal region of the receptor protein, it is preferable to use the-antibody recognizing the region other than the C-terminal region for the primary reaction, e.g., the antibody recognizing the N-terminal region.

The monoclonal antibodies of the present invention can be used for the assay systems other than the sandwich method, for example, competitive method, immunometric method, nephrometry, etc. In the competitive method, antigen in a test fluid and the labeled antigen are competitively reacted with antibody, and the unreacted labeled antigen (F) and the labeled antigen bound to the antibody (B) are separated (B/F separation). The amount of the label in B or F is measured, and the amount of the antigen in the test fluid is quantified. This reaction method includes a liquid phase method using a soluble antibody as an antibody, polyethylene glycol for B/F separation and a secondary antibody to the soluble antibody, and an immobilized method either using an immobilized antibody as the primary antibody, or using a soluble antibody as the primary antibody and immobilized antibody as the secondary antibody.

In the immunometric method, antigen in a test fluid and immobilized antigen are competitively reacted with a definite amount of labeled antibody, the immobilized phase is separated from the liquid phase, or antigen in a test fluid and an excess amount of labeled antibody are reacted, immobilized antigen is then added to bind the unreacted labeled antibody to the immobilized phase, and the immobilized phase is separated from the liquid phase. Then, the amount of the label in either phase is measured to quantify the antigen in the test fluid.

In the nephrometry, insoluble precipitate produced after the antigen-antibody reaction in gel or solution is quantified. When the amount of antigen in the test fluid is small and only a small amount of precipitate is obtained, laser nephrometry using scattering of laser is advantageously employed.

For applying these immunological methods to the measurement methods of the present invention, any particular conditions or procedures are not required. Systems for measuring the protein of the present invention or its salts are constructed by adding the usual technical consideration in the art to the conventional conditions and procedures. For the details of these general technical means, reference can be made to the following reviews and texts. For example, Hiroshi Irie, ed. “Radioimmunoassay” (Kodansha, published in 1974), Hiroshi Irie, ed. “Sequel to the Radioimmunoassay” (Kodansha, published in 1979), Eiji Ishikawa, et al. ed. “Enzyme immonoassay” (Igakushoin, published in 1978), Eiji Ishikawa, et al. ed. “Immunoenzyme assay” (2nd ed.) (Igakushoin, published in 1982), Eiji Ishikawa, et al. ed. “Immunoenzyme assay” (3rd ed.) (Igakushoin, published in 1987), Methods in ENZYMOLOGY, Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays)), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies))(all published by Academic Press Publishing).

As described above, the protein of the present invention or its salts can be quantified with high sensitivity, using the antibodies of the present invention. By quantifying the protein of the present invention or its salts using the antibodies of the present invention, diagnosis can be made on various diseases associated with dysfunction of the protein of the present invention.

The antibodies of the present invention can also be used for specifically detecting the protein etc. of the present invention present in test samples such as body fluids or tissues. The antibodies may also be used for preparation of antibody columns for purification of the protein etc. of the present invention, for detection of the protein etc. of the present invention in each fraction upon purification, and for analysis of the behavior of the protein of the present invention in the test cells.

(7) Preparation of Non-Human Transgenic Animals Carrying the DNA Encoding the G Protein-Coupled Protein of the Present Invention

Using the DNA of the present invention, non-human transgenic animals expressing the protein etc. of the present invention can be prepared. Examples of the non-human animals include mammals (e.g., rats, mice, rabbits, sheep, swine, bovine, cats, dogs, monkeys, etc.) (hereinafter referred to as animals) can be used, with mice and rabbits being particularly appropriate.

To transfer the DNA of the present invention to target animals, it is generally advantageous to use the DNA in a gene construct ligated downstream of a promoter that can express the DNA in animal cells. For example, when the DNA of the present invention derived from rabbit is transferred, e.g., the gene construct, in which the DNA is ligated downstream of a promoter that can expresses the DNA of the present invention derived from animals containing the DNA of the present invention highly homologous to the rabbit-derived DNA, is microinjected to rabbit fertilized ova; thus, the DNA-transferred animal, which is capable of producing a high level of the protein etc. of the present invention, can be prepared. Examples of the promoters that are usable include virus-derived promoters and ubiquitous expression promoters such as metallothionein promoter, but promoters of NGF gene and enolase that are specifically expressed in the brain are preferably used.

The transfer of the DNA of the present invention at the fertilized egg cell stage secures the presence of the DNA in all germ and somatic cells in the prepared animal. The presence of the protein etc. of the present invention in the germ cells in the DNA-transferred animal means that all germ and somatic cells contain the protein etc. of the present invention in all progenies of the animal. The progenies of the animal that took over the gene contain the protein etc. of the present invention in all germ and somatic cells.

The DNA-transferred animals of the present invention can be maintained and bled in the conventional environment as animals carrying the DNA after confirming the stable retention of the gene in the animals through mating. Furthermore, mating male and female animals containing the objective DNA results in acquiring homozygous animals having the transferred gene on both homologous chromosomes. By mating the male and female homozygotes, bleeding can be performed so that all progenies contain the DNA.

Since the protein etc. of the present invention is highly expressed in the animals in which the DNA of the present invention has been transferred, the animals are useful for screening of agonists or antagonists to the protein etc. of the present invention.

The animals in which the DNA of the present invention has been transferred can also be used as cell sources for tissue culture. The protein of the present invention can be analyzed by, for example, directly analyzing the DNA or RNA in tissues from the mouse in which the DNA of the present invention has been transferred, or by analyzing tissues containing the protein etc. expressed from the gene. Cells from tissues having the protein etc. of the present invention are cultured by the standard tissue culture technique. Using these cells, for example, the function of tissue cells such as cells derived from the brain or peripheral tissues, which are generally difficult to culture, can be studied. Using these cells, for example, it is possible to select pharmaceuticals that increase various tissue functions. When a highly expressing cell line is available, the protein etc. of the present invention can be isolated and purified from the cell line.

In the specification and drawings, the codes of bases and amino acids are denoted in accordance with the IUPAC-IUB Commission on Biochemical Nomenclature or by the common codes in the art, examples of which are shown below. For amino acids that may have the optical isomer, L form is presented unless otherwise indicated.

DNA: deoxyribonucleic acid

cDNA: complementary deoxyribonucleic acid

A: adenine

T: thymine

G: guanine

C: cytosine

RNA: ribonucleic acid

mRNA: messenger ribonucleic acid

dATP: deoxyadenosine triphosphate

dTTP: deoxythymidine triphosphate

dGTP: deoxyguanosine triphosphate

dCTP: deoxycytidine triphosphate

Gly or G: glycine

Ala or A: alanine

Val or V: valine

Leu or L: leucine

Ile or I: isoleucine

Ser or S: serine

Thr or T: threonine

Cys or C: cysteine

Met or M: methionine

Glu or E: glutamic acid

Asp or D: aspartic acid

Lys or K: lysine

Arg or R: arginine

His or H: histidine

Phe or F: phenylalanine

Tyr or Y: tyrosine

Trp or W: tryptophan

Pro or P: proline

Asn or N: asparagine

Gln or Q: glutamine

pGlu: pyroglutamic acid

Xaa: unidentified amino acid residue

Tos: p-toluenesulfonyl

Bzl: benzyl

Cl₂Bl: 2,6-dichlorobenzyl

Bom: benzyloxymethyl

Z: benzyloxycarbonyl

Cl-Z: 2-chlorobenzyloxycarbonyl

Br-Z: 2-bromobenzyloxycarbonyl

Boc: t-butoxycarbonyl

DNP: dinitrophenol

Trt: trityl

Bum: t-butoxymethyl

Fmoc: N-9-fluorenylmethoxycarbonyl

HOBt: 1-hydroxybenztriazole

HOOBt: 3,4-dihydro-3-hydroxy-4-oxo-1,2,3-benzotriazine

HONB: 1-hydroxy-5-norbornene-2,3-dicarboximide

DCC: N,N′-dicyclohexylcarbodiimide

ATP: adenosine triphosphate

EDTA: ethylenediamine tetraacetic acid

SDS: sodium dodecyl sulfate

The sequence identification numbers in the sequence listing of the specification indicates the following sequence, respectively.

[SEQ ID NO: 1]

This shows the base sequence of primer used in Example 1 described later.

[SEQ ID NO: 2]

This shows the base sequence of primer used in Example 1 described later.

[SEQ ID NO: 3]

This shows the base sequence of cDNA encoding the novel G protein-coupled receptor protein of the present invention (rZAQ1).

[SEQ ID NO: 4]

This shows the amino acid sequence of the novel G protein-coupled receptor protein of the present invention (rZAQ1).

[SEQ ID NO: 5]

This shows the base sequence of probe used in Example 2 described later.

[SEQ ID NO: 6]

This shows the base sequence of probe used in Example 2 described later.

[SEQ ID NO: 7]

This shows the base sequence of primer used in Example.2 described later.

[SEQ ID NO: 8]

This shows the base sequence of primer used in Example 2 described later.

[SEQ ID NO: 9]

This shows the base sequence of primer used in Example 2 described later.

[SEQ ID NO: 10]

This shows the base sequence of cDNA encoding the novel G protein-coupled receptor protein of the present invention (rZAQ2). [SEQ ID NO: 11]

This shows the amino acid sequence of the novel G protein-coupled receptor protein of the present invention (rZAQ2).

[SEQ ID NO: 12]

This shows the amino acid sequence of human brain-derived G protein-coupled receptor protein (ZAQ).

[SEQ ID NO: 13]

This shows the base sequence of primer 1 used in Example 3 described later.

[SEQ ID NO: 14]

This shows the base sequence of primer 2 used in Example 3 described later.

[SEQ ID NO: 15]

This shows the base sequence of rZAQ1 probe used in Example 3 described later.

[SEQ ID NO: 16]

This shows the base sequence of primer 3 used in Example 3 described later.

[SEQ ID NO: 17]

This shows the base sequence of primer 4 used in Example 3 described later.

[SEQ ID NO: 18]

This shows the base sequence of rZAQ2 used in Example 3 described later.

[SEQ ID NO: 19]

This shows the base sequence of primer rZAQ1Sal used in Example 3 described later.

[SEQ ID NO: 20]

This shows the base sequence of primer rZAQ1Spe used in Example 3 described later.

[SEQ ID NO: 21]

This shows the base sequence of primer rZAQ2Sal used in Example 3 described later.

[SEQ ID NO: 22]

This shows the base sequence of primer rZAQ2Spe used in Example 3 described later.

[SEQ ID NO: 23]

This shows the base sequence of primer used in Example 4 described later.

[SEQ ID NO: 24]

This shows the base sequence of primer used in Example 4 described later.

[SEQ ID NO: 25]

This shows the base sequence of primer used in Example 4 described later.

[SEQ ID NO: 26]

This shows the base sequence of primer used in Example 4 described later.

[SEQ ID NO: 27]

This shows the base sequence of DNA fragment obtained in Example 4 described later.

[SEQ ID NO: 28]

This shows the base sequence of DNA fragment obtained in Example 4 described later.

[SEQ ID NO: 29]

This shows the base sequence of primer used in Example 4 described later.

[SEQ ID NO: 30]

This shows the base sequence of primer used in Example 4 described later.

[SEQ ID NO: 31]

This shows the base sequence of primer used in Example 4 described later.

[SEQ ID NO: 32]

This shows the base sequence of 5′ end of the DNA encoding rat ZAQ ligand peptide obtained in Example 4 described later.

[SEQ ID NO: 33]

This shows the base sequence of primer used in Example 4 described later.

[SEQ ID NO: 34]

This shows the base sequence of primer used in Example 4 described later.

[SEQ ID NO: 35]

This shows the base sequence of 5′ end of the DNA encoding rat ZAQ ligand peptide obtained in Example 4 described later.

[SEQ ID NO: 36]

This shows the base sequence of primer used in Example 4 described later.

[SEQ ID NO: 37]

This shows the base sequence of primer used in Example 4 described later.

[SEQ ID NO: 38]

This shows the base sequence of primer used in Example 4 described later.

[SEQ ID NO: 39]

This shows the base sequence of primer used in Example 4 described later.

[SEQ ID NO: 40]

This shows the base sequence of DNA fragment obtained in Example 4 described later (normal type).

[SEQ ID NO: 41]

This shows the base sequence of DNA fragment obtained in Example 4 described later (Y type).

[SEQ ID NO: 42]

This shows the base sequence of DNA fragment obtained in Example 4 described later (Q type).

[SEQ ID NO: 43]

This shows the amino acid sequence of rat ZAQ ligand precursor peptide (normal type).

[SEQ ID NO: 44]

This shows the base sequence of the DNA encoding rat ZAQ ligand precursor peptide (normal type).

[SEQ ID NO: 45]

This shows the amino acid sequence of rat ZAQ ligand precursor peptide (Y type).

[SEQ ID NO: 46]

This shows the base sequence of the DNA encoding rat ZAQ ligand precursor peptide (Y type).

[SEQ ID NO: 47]

This shows the amino acid sequence of rat ZAQ ligand precursor peptide (Q type).

[SEQ ID NO: 48]

This shows the base sequence of the DNA encoding rat ZAQ ligand precursor peptide (Q type).

[SEQ ID NO: 49]

This shows the amino acid sequence of rat ZAQ ligand mature peptide (normal type).

[SEQ ID NO: 50]

This shows the base sequence of the DNA encoding rat ZAQ ligand mature peptide (normal type).

[SEQ ID NO: 51]

This shows the amino acid sequence of rat ZAQ ligand mature peptide (Y type).

[SEQ ID NO: 52]

This shows the base sequence of the DNA encoding rat ZAQ ligand mature peptide (Y type).

[SEQ ID NO: 53]

This shows the amino acid sequence of rat-ZAQ ligand mature peptide (Q type).

[SEQ ID NO: 54]

This shows the base sequence of the DNA encoding rat ZAQ ligand mature peptide (Q type).

[SEQ ID NO: 55]

This shows the base sequence of primer BF2 used in Example 5 described later.

[SEQ ID NO: 56]

This shows the base sequence of primer BR1 used in Example 5 described later.

[SEQ ID NO: 57]

This shows the base sequence of DNA fragment obtained in Example 5 described later.

[SEQ ID NO: 58]

This shows the base sequence of primer RB5-1 used in Example 5 described later.

[SEQ ID NO: 59]

This shows the base sequence of primer RB5-3 used in Example 5 described later.

[SEQ ID NO: 60]

This shows the base sequence of 5′ end of the DNA encoding rat Bv8 obtained in Example 5 described later.

[SEQ ID NO: 61]

This shows the base sequence of primer RB3-1 used in Example 5 described later.

[SEQ ID NO: 62]

This shows the base sequence of primer RB3-2 used in Example 5 described later.

[SEQ ID NO: 63]

This shows the base sequence of 3′ end of the DNA encoding rat Bv8 obtained in Example 5 described later.

[SEQ ID NO: 64]

This shows the base sequence of primer RBv8-WF1 used in Example 5 described later.

[SEQ ID NO: 65]

This shows the base sequence of primer RBv8-WF2 used in Example 5 described later.

[SEQ ID NO: 66]

This shows the base sequence of primer RBv8-WR1 used in Example 5 described later.

[SEQ ID NO: 67]

This shows the base sequence of primer RBv8-WR2 used in Example 5 described later.

[SEQ ID NO: 68]

This shows the base sequence of DNA fragment obtained in Example 5 described later.

[SEQ ID NO: 69]

This shows the amino acid sequence of rat Bv8 precursor peptide.

[SEQ ID NO: 70]

This shows the base sequence of the DNA encoding rat Bv8 precursor peptide.

[SEQ ID NO: 71]

This shows the amino acid sequences of rat Bv8 mature peptide and mouse Bv8 mature peptide.

[SEQ ID NO: 72]

This shows the base sequence of the DNA encoding rat Bv8 mature peptide.

The transformant Escherichia coli DH5α/pCR2.1-rZAQ1 obtained in Example 1 described below was on deposit with International Patent Organisms Depository, National Institute of Advanced Industrial Science and Technology (formerly, National Institute of Bioscience and Human-Technology (NIBH), Ministry of International Trade and Industry), located at Central 6, 1-1-1 Higashi, Tsukuba, Ibaraki, 305-8566, Japan, as the Accession Number FERM BP-7275 on Aug. 21, 2000 and with Institute for Fermentation (IFO), located at 2-17-85 Juso-honmachi, Yodogawa-ku, Osaka-shi, Osaka, 532-8686, Japan, as the Accession Number IFO 16459 on Aug. 1, 2000.

The transformant Escherichia coli DH10B/pCMV-rZAQ2 obtained in Example 2 described below was on deposit with International Patent Organisms Depository, National Institute of Advanced Industrial Science and Technology (formerly, National Institute of Bioscience and Human-Technology (NIBH), Ministry of International Trade and Industry), located at Central 6, 1-1-1 Higashi, Tsukuba, Ibaraki, 305-8566, Japan, as the Accession Number FERM BP-7276 on Aug. 21, 2000 and with Institute for Fermentation (IFO), located at 2-17-85 Juso-honmachi, Yodogawa-ku, Osaka-shi, Osaka, 532-8686, Japan, as the Accession Number IFO 16460 on Aug. 1, 2000.

The transformant Escherichia coli TOP10/pRMIT obtained in Example 4 described below was on deposit with National Institute of Bioscience and Human-Technology, Ministry of Economy, Trade and Industry (formerly, National Institute of Bioscience and Human-Technology (NIBH), Ministry of International Trade and Industry), located at Central 6, 1-1-1 Higashi, Tsukuba, Ibaraki, 305-8566, Japan, as the Accession Number FERM BP-7426 on Jan. 11, 2001 and with Institute for Fermentation (IFO), located at 2-17-85 Juso-honmachi, Yodogawa-ku, Osaka-shi, Osaka, 532-8686, Japan, as the Accession Number IFO 16521 on Dec. 22, 2000.

The transformant Escherichia coli TOP10/pRBv obtained in Example 5 described below was on deposit with International Patent Organisms Depository, National Institute of Advanced Industrial Science and Technology (formerly, National Institute of Bioscience and Human-Technology (NIBH), Ministry of Economy, Trade and Industry), located at Central 6, 1-1-1 Higashi, Tsukuba, Ibaraki, 305-8566, Japan, as the Accession Number FERM BP-7427 on Jan. 11, 2001 and with Institute for Fermentation (IFO), located at 2-17-85 Juso-honmachi, Yodogawa-ku, Osaka-shi, Osaka, 532-8686, Japan, as the Accession Number IFO 16522 on Dec. 22, 2000.

### EXAMPLES

The present invention is described in detail below with reference to EXAMPLES, but is not deemed to limit the scope of the present invention thereto. The gene manipulation procedures using Escherichia coli were performed according to the methods described in the Molecular Cloning.

### Example 1

### Cloning of the cDNA Encoding the Novel Rat Brain-Derived G Protein-Coupled Receptor Protein (rZAQ1) and Determination of the Base Sequence

Using rat whole brain-derived cDNA (CLONTECH) as a template and two primers (SEQ ID NO: 1 and SEQ ID NO: 2), PCR was carried out. The reaction solution in the above reaction comprised of 1/10 volume of the above cDNA as a template, 1/50 volume of Advantage 2 cDNA Polymerase Mix (CLONTECH), 0.2 μM each of primers, 200 μM of dNTPs, and a buffer attached to the enzyme to make the total volume 25 μl. The PCR reaction was carried out by reaction of 94° C. for 2 minutes, then a cycle set to include 94° C. for 20 seconds followed by 72° C. for 1 minute and 30 seconds, which was repeated 3 times, 94° C. for 20 seconds followed by 68° C. for 1 minute and 30 seconds, which was repeated 3 times, 94° C. for 20 seconds followed by 62° C. for 20 seconds and 68° C. for 1 minute, which was repeated 36 times, and finally, extension reaction at 68° C. for 7 minutes. The PCR product was subcloned to plasmid vector pCR2.1-TOPO (Invitrogen Inc.) following the instructions attached to the TOPO-TA Cloning Kit (Invitrogen Inc.). The plasmid was then introduced into Escherichia coli DH5α, and the clones containing the cDNA were selected on LB agar plates containing ampicillin. As a result of analysis for sequence of each clone, cDNA sequence encoding the novel G protein-coupled receptor protein was obtained (SEQ ID NO: 3). It was observed that an amino acid sequence deduced from the base sequence of the cDNA (SEQ ID NO: 4) has 83.7% homology to the amino acid sequence of ZAQ, the human brain-derived G protein-coupled receptor protein (WO 01/16309) (SEQ ID NO: 12). The novel G protein-coupled receptor protein containing the amino acid sequence was designated rZAQ1. The transformant (E. coli) containing the DNA having the base sequence represented by SEQ ID NO: 3 was designated Escherichia coli DH5α/pCR2.1-rZAQ1.

### Example 2

### Cloning of the cDNA Encoding the Novel Rat Brain-Derived G Protein-Coupled Receptor Protein (rZAQ2) and Determination of the Base Sequence

The clone encoding rZAQ2 was acquired by the gene trapper method. That is, after biotinylation of the probe (SEQ ID NO: 5 and SEQ ID NO: 6), the biotinylated probe was hybridized with a denatured rat whole brain-derived cDNA library (GIBCO-BRL) to acquire a single-stranded gene. The obtained single-stranded gene was repaired to double strand using primers (SEQ ID NO: 7 and SEQ ID NO: 8). The gene was electroporated into Escherichia coli DH10B to get transformants using ampicillin resistance as an index. Further, the clones encoding the objective base sequence were selected by colony PCR using probe (SEQ ID NO: 5) and primer (SEQ ID NO: 9). It was observed that an amino acid sequence (SEQ ID NO: 11) deduced from the base sequence of ORF (open reading frame) (SEQ ID NO: 10) predicted from the base sequence of the clone has 80.6% homology to rZAQ1. The novel G protein-coupled receptor protein containing the amino acid sequence was designated rZAQ2. The transformant (E. coli) acquired by the gene trapper method was designated Escherichia coli DH10B/pCMV-rZAQ2.

### Example 3

### Analysis of Distribution of Expression for rZAQ1 and rZAQ2 by Using Taqman PCR

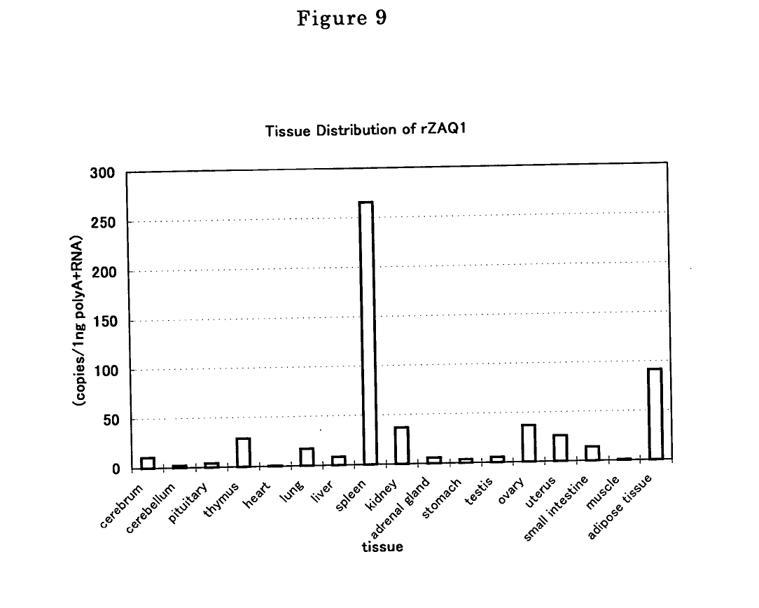

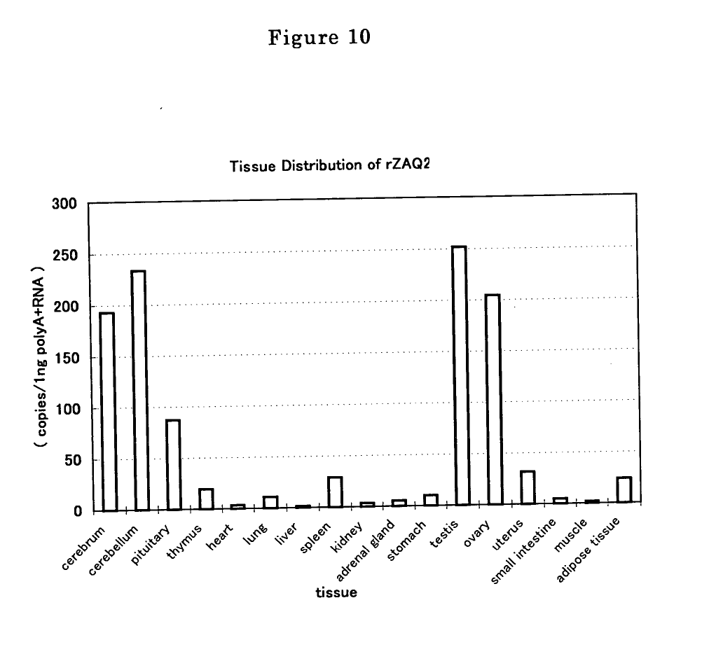

A probe and primers used for Taqman PCR were retrieved by using Primer Express ver. 1.0 (PE Biosystems Japan). Primer 1 (SEQ ID NO: 13), primer 2 (SEQ ID NO: 14) and rZAQ1 probe (SEQ ID NO: 15) were selected from the base sequence of rZAQ1. From the base sequence of rZAQ2, primer 3 (SEQ ID NO: 16), primer 4 (SEQ ID NO: 17) and rZAQ2 probe (SEQ ID NO: 18) were selected. As a reporter dye for probe, FAM (6-carboxyfluorescein) was added to the probe. For standard DNAs, two DNAs were prepared as follows. That is, the rZAQ1 was amplified by PCR using pCR2.1-rZAQ1 (EXAMPLE 1) as a template, and primers rZAQ1Sal (SEQ ID NO: 19) and rZAQ1Spe (SEQ ID NO: 20). The rZAQ2 was amplified by PCR using pCR2.1-rZAQ2 (EXAMPLE 2) as a template, and primers rZAQ2Sal (SEQ ID NO: 21) and rZAQ2Spe (SEQ ID NO: 220). As a result, each-fragment was prepared. Each fragment was purified with CHROMA SPIN200 (CLONTECH Laboratories, Inc. (CA, USA)) and adjusted to 10⁰-10⁶ copies/μl for use. As a cDNA source of each tissue, 0.5-1.0 g each of 21 tissues (cerebrum, cerebellum, pituitary, spinal cord, thymus, heart, lung, liver, spleen, kidney, adrenal, stomach, testis, ovary, uterus, small intestine, colon, caecum, pancreas, skeletal muscle, adipose cell; for ovary and uterus, collected from female rat) was collected from Wistar rat (male or female, 7.5 week age, Charles River Japan, Inc.). Then, total RNA was extracted from the tissues using TRIZOL reagent (Gibco BRL) in accordance with the method described in the attached manual. Next, poly(A)⁺ RNA was prepared from the above-mentioned total RNA using oligo-dT cellulose column (MessageMaker reagent assembly, Gibco BRL, or the equivalent product, mRNA purification kit, Pharmacia for ovary and uterus) according to the method described the attached manual. Further, first strand cDNA was synthesized from 500 ng of poly(A)⁺ RNA described above at 42° C. in 20 μl of the reaction volume using SuperScript Preamplification System for First Strand cDNA Synthesis (Gibco BRL) and oligo(dT) primer according to the method described in the attached manual. After a set amount of Taqman Universal PCR Master Mix (PE Biosystems Japan) described in the attached document was added to primers, a probe and a template, PCR reaction and analysis were performed with ABI PRISM 7700 Sequence Detection System (PE Biosystems Japan). The result is converted to copies per 1 ng of poly(A)⁺ RNA-used at the starting point of cDNA synthesis, and then shown in FIG. 9 and FIG. 10.

The highly expression of rZAQ1 was observed in spleen, adipose cells and the like. The highly expression of rZAQ2 was observed in testis, ovary and central nerve.

### Example 4

### Cloning of cDNA of Rat ZAQ Ligand Peptide

Using rat brain QUICK-clone cDNA (CLONTECH) as a template, degenerate primers MF1 (SEQ ID NO: 23), MR1 (SEQ ID NO: 24), MF2 (SEQ ID NO: 25) and MR2 (SEQ ID NO: 26) were prepared. Then the PCR reaction described below was carried out.

| | |
|---|---|
| MF1: | |
| 5′-TCACCYCAAGTGAYCATGAGAGG-3′ | (SEQ ID NO: 23) |
| MR1: | |
| 5′-CTAAAARTTGRYRTTCTTCAAGTCC-3′ | (SEQ ID NO: 24) |
| MF2: | |
| 5′-ATCACAGGGGCCTGTGARCG-3′ | (SEQ ID NO: 25) |
| MR2: | |
| 5′-AGCAGCGGTACCTGCCGTCC-3′ | (SEQ ID NO: 26) |

PCR reaction solution was prepared by admixing 0.6 μl of 50× Advantage 2 Polymerase Mix (CLONTECH), 3 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 2.4 μl of dNTP mixture (2.5 mM each, Takara Shuzo), 0.6 μl of 10 μM primers MF1 and MR1, 1 μl of template cDNA, and 20.6 μl of distilled water. The reaction condition was 95° C. for 1 minute for the starting denaturation, then a cycle set to include 95° C. for 30 seconds followed by 55° C. for 1 minute and 68° C. for 1 minute, which was repeated 35 times, and finally, extension reaction at 68° C. for 5 minutes.

Next, using a solution, wherein the PCR reaction solution described above was 15-fold diluted with distilled water, as a template, nested PCR was performed. PCR reaction solution was prepared by admixing 0.6 μl of 50× Advantage 2 Polymerase Mix (CLONTECH), 3 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 2.4 μl of dNTP mixture (2.5 mM each, Takara Shuzo), 0.6 μl of 10 μM primers MF1 and MR1, 1 μl of template cDNA, and 20.6 μl of distilled water. The reaction condition was 95° C. for 1 minute for the starting denaturation, then a cycle set to include 95° C. for 30 seconds followed by 55° C. for 1 minute and 68° C. for 1 minute, which was repeated 35 times, and finally, extension reaction at 68° C. for 5 minutes.

The DNA fragment obtained was cloned in accordance with the method described in the attached manual using the Zero Blunt TOPO PCR Cloning Kit (Invitrogen). The cloned DNA sequence was decoded by using ABI377 DNA sequencer, thereby partial sequence represented by SEQ ID NO: 27 (T type) and SEQ ID NO: 28 (C type) were obtained.

From the above-mentioned information on the sequence, primers RM3-1 (SEQ ID NO: 29), RM3-2 (SEQ ID NO: 30) and RM3-3 (SEQ ID NO: 31) were prepared. Then the 5′ RACE experiment described below was carried out.

| | |
|---|---|
| RM3-1: | |
| 5′-GTGGCACTCCTCTCCTTCCCGCCCCAGA-3′ | (SEQ ID NO: 29) |
| RN3-2: | |
| 5′-CAGGCCCCGCAGCCACAGGCTGATAGCA-3′ | (SEQ ID NO: 30) |
| RM3-3: | |
| 5′-AGCAGGTGCCAGCCCCACACTGGACATC-3′ | (SEQ ID NO: 31) |

PCR reaction solution of 5′ RACE was prepared by admixing 0.6 μl of 50× Advantage 2 Polymerase Mix (CLONTECH), 6 μl of attached 5× Advantage-GC 2 PCR buffer (200 mM Tricine-KOH, 75 mM KOAc, 17.5 mM Mg(OAc)₂, 25% Dimethyl Sulfoxide, 18.75 μg/ml BSA, 0.025% Tween-20, 0.025% Nonidet-P40), 2.4 μl of dNTP mixture (2.5 mM each, Takara Shuzo), 0.6 μl of 10 μM primer RM3-1, 0.6 μl of 10 μM primer AP1 (the primer AP1 is attached with Marathon Ready cDNA Kit by CLONTECH), 3 μl of template cDNA (CLONTECH, rat brain Marathon Ready cDNA), and 16.8 μl of distilled water. The reaction condition was 94° C. for 30 seconds for the starting denaturation, then a cycle set to include 94° C. for 5 seconds followed by 72° C. for 3 minute, which was repeated 5 times, 94° C. for 5 seconds followed by 70° C. for 3 minute, which was repeated 5 times, and finally, 94° C. for 5 seconds followed by 68° C. for 3 minute, which was repeated 25 times.

Next, using the PCR reaction solution described above as a template, nested PCR was performed. The reaction solution was prepared by admixing 0.6 μl of 50× Advantage 2 Polymerase Mix (CLONTECH), 6 μl of attached 5× Advantage 2 PCR buffer (200 mM Tricine-KOH, 75 mM KOAc, 17.5 mM Mg(OAc)₂, 25% Dimethyl Sulfoxide, 18.75 μg/ml BSA, 0.025% Tween-20, 0.025% Nonidet-P40), 2.4 μl of dNTP mixture (2.5 mM each, Takara Shuzo), 0.6 μl of 10 μM primer RM3-2 or RM3-3, 0.6 μl of 10 μM primer AP2 (the primer AP2 is attached with Marathon Ready cDNA Kit by CLONTECH), 3 μl of template cDNA (the PCR reaction solution diluted to 50 fold), and 16.8 μl of distilled water. The reaction condition was 94° C. for 30 seconds for the starting denaturation, then a cycle set to include 94° C. for 5 seconds followed by 68° C. for 3 minute, which was repeated 30 times.

The DNA fragment obtained was cloned in accordance with the method described in the attached manual using the TOPO TA Cloning Kit (Invitrogen). The cloned DNA sequence was decoded by using ABI377 DNA sequencer, thereby the 5′ sequence (SEQ ID NO: 32) was obtained.

From the information of SEQ ID NO: 27 and SEQ ID NO: 28, primers RM5-1 (SEQ ID NO: 33) and RM5-4 (SEQ ID NO: 34) were prepared. Then the 3′ RACE experiment described below was carried out.

| | |
|---|---|
| RM5-1: | |
| 5′-GGAAGGAGAGGAGTGCCACCCTGGAAG-3′ | (SEQ ID NO: 33) |
| RM5-4: | |
| 5′-ACCATACCTGTCCCTGTTCACCCAGCCT-3′ | (SEQ ID NO: 34) |

PCR reaction solution of 3′ RACE was prepared by admixing 0.6 μl of 50× Advantage 2 Polymerase Mix (CLONTECH), 2.4 μl of attached 5× Advantage-GC 2 PCR buffer (200 mM Tricine-KOH, 75 mM KOAc, 17.5 mM Mg(OAc)₂, 25% Dimethyl Sulfoxide, 18.75 μg/ml BSA, 0.025% Tween-20, 0.025% Nonidet-P40), 2.4 μl of dNTP mixture (2.5 mM each, Takara Shuzo), 0.6 μl of 10 μM primer RM5-1, 0.6 μl of 10 μM primer AP1 (the primer AP1 is attached with Marathon Ready cDNA Kit by CLONTECH), 3 μl of template cDNA (CLONTECH, rat brain Marathon Ready cDNA), and 16.8 μl of distilled water. The reaction condition was 94° C. for 1 minute for the starting denaturation, then a cycle set to include 94° C. for 30 seconds followed by 72° C. for 3 minute, which was repeated 5 times, 94° C. for 30 seconds followed by 70° C. for 3 minute, which was repeated 5 times, 94° C. for 30 seconds followed by 68° C. for 3 minute, which was repeated 25 times, and finally, extension reaction at 68° C. for 3 minutes.

Next, using the PCR reaction solution described above as a template, nested PCR was performed. The reaction solution was prepared by admixing 0.6 μl of 50× Advantage 2 Polymerase Mix (CLONTECH), 6 μl of attached 5× Advantage 2 PCR buffer (200 mM Tricine-KOH, 75 mM KOAc, 17.5 mM Mg(OAc)₂, 25% Dimethyl Sulfoxide, 18.75 μg/ml BSA, 0.025% Tween-20, 0.025% Nonidet-P40), 2.4 μl of dNTP mixture (2.5 mM each, Takara Shuzo), 0.6 μl of 10 μM primer RM5-4 or RM3-3, 0.6 p1 of 10 μM primer AP2 (the primer AP2 is attached with Marathon Ready cDNA Kit by CLONTECH), 3 μl of template cDNA (the PCR reaction solution diluted to 50 fold), and 16.8 μl of distilled water. The reaction condition was 94° C. for 1 minute for the starting denaturation, then a cycle set to include 94° C. for 30 seconds followed by 68° C. for 3 minute, which was repeated 35 times, and finally, extension reaction at 68° C. for 3 minutes.

The DNA fragment obtained was cloned in accordance with the method described in the attached manual using the TOPO TA Cloning Kit (Invitrogen). The cloned DNA sequence was decoded by using ABI377 DNA sequencer, thereby the 3′ sequence (SEQ ID NO: 35) was obtained.

From the information of 5′ RACE and 3′ RACE, primers RBv8-WF1 (SEQ ID NO: 36), RBv8-WF2 (SEQ ID NO: 37), RBv8-WR1 (SEQ ID NO: 38) and RBv8-R2 (SEQ ID NO: 39) were prepared using rat brain QUICK-clone cDNA (CLONTECH) or rat brain cDNA (Wistar rat) as a template. Then the PCR reaction described below was carried out.

(SEQ ID NO: 36)

| | |
|---|---|
| RMIT-WF1: | 5′-ATTCCAGAGTGGACAGTGTTTGCCTTCACC-3′ |

(SEQ ID NO: 37)

| | |
|---|---|
| RMIT-WF2: | 5′-GATCATGAGAGGTGCTGTGCAAGTCTTC-3′ |

(SEQ ID NO: 38)

| | |
|---|---|
| RMIT-WR1: | 5′-CTCTCTGCACGCTGCTGGACTGTTCC-3′ |

(SEQ ID NO: 39)

| | |
|---|---|
| RMIT-WR2: | 5′-CAGATGTAACACAAGAGGTCACCCAGTAGG-3′ |

The PCR reaction solution was prepared by admixing 0.6 μl of PfuTurbo DNA polymerase (Stratagene), 3 μl of attached 10× PCR buffer, 2.4 μl of 2.5 mM dNTP mixture, 1.5 μl each of 10 μM primers RMIT-WF1 and RMIT-WR1, 1 μl of template DNA and 20 μl of distilled water. The reaction condition was 95° C. for 1 minute for the starting denaturation, then a cycle set to include 95° C. for 30 seconds followed by 55° C. for 30 seconds and 72° C. for 1 minute, which was repeated 35 times, and finally, extension reaction at 72° C. for 5 minutes.

Next, using a solution, wherein the PCR reaction solution described above was 50-fold diluted with distilled water, as a template, nested PCR was performed. The PCR reaction solution was prepared by admixing 0.6 μl of PfuTurbo DNA polymerase (Stratagene), 3 μl of attached 10× PCR buffer, 2.4 μl of 2.5 mM dNTP mixture, 1.5 μl each of 10 μM primers RMIT-WF1 and RMIT-WR1, 3 μl of template DNA and 18 μl of distilled water. The reaction condition was 95° C. for 1 minute for the starting denaturation, then a cycle set to include 95° C. for 30 seconds followed by 55° C. for 30 seconds and 72° C. for 1 minute, which was repeated 35 times, and finally, extension reaction at 72° C. for 5 minutes.

The DNA fragment obtained was cloned in accordance with the method described in the attached manual using the Zero Blunt TOPO PCR Cloning Kit (Invitrogen). The cloned DNA sequence was decoded by using ABI377 DNA sequencer, then plasmids having 3 kinds of 375 bp DNA fragment encoding rat ZAQ ligand entire peptide (SEQ ID NO: 40, SEQ ID NO: 41 and SEQ ID NO: 42; the sequences occurring base substitution denote normal type, Y type and Q type, respectively) were designated pRMIT, pRMITY and pRMITQ, respectively. Escherichia coli TOP10 was transformed by the plasmids, then the transformants obtained were designated E. coli TOP10/pRMIT, E. coli TOP10/pRMITY and E. coli TOP1.0/pRMITQ, respectively.

From a result of analyzing the base sequence of these DNA fragments, it was clarified that the DNA fragment represented by SEQ ID NO: 40, the DNA fragment represented by SEQ ID NO: 41 and the DNA fragment represented by SEQ ID NO: 42 contain a DNA (SEQ ID NO: 44) encoding rat ZAQ ligand precursor peptide (105 amino acid residues) represented by SEQ ID NO: 43, a DNA (SEQ ID NO: 46) encoding rat ZAQ ligand precursor peptide (105 amino acid residues) represented by SEQ ID NO: 45 and a DNA (SEQ ID NO: 48) encoding rat ZAQ ligand precursor peptide (105 amino acid residues) represented by SEQ ID NO: 47, respectively.

In addition, the base sequences represented by SEQ ID NO: 43, SEQ ID NO: 45 and SEQ ID NO: 47 have a typical signal sequence. Moreover, it became clear that the DNA having a base sequence represented by SEQ ID-NO: 43, the DNA having a base sequence represented by SEQ ID NO: 45 and the DNA having a base sequence represented by SEQ ID NO: 47 contain a 258 bp DNA (SEQ ID NO: 50) encoding rat ZAQ ligand mature peptide (86 amino acid residues) represented by SEQ ID NO: 49, a 258 bp DNA (SEQ ID NO: 52) encoding rat ZAQ ligand mature peptide (86 amino acid residues) represented by SEQ ID NO: 51 and a 258 bp DNA (SEQ ID NO: 54) encoding rat ZAQ ligand mature peptide (86 amino acid residues) represented by SEQ ID NO: 53, respectively.

In the amino acid sequence represented by SEQ ID NO: 49, His at the position of 46 in SEQ ID NO: 51 and Arg at the position of 36 in SEQ ID NO: 53 were substituted to Tyr and Gln, respectively.

### Example 5

### Cloning of cDNA of Rat Bv8 Peptide

Using rat testis Marathon-Ready cDNA (CLONTECH) as a template, degenerate primer BF2 (SEQ ID NO: 55) and primer BR1 (SEQ ID NO: 56) were prepared. Then the PCR reaction described below was carried out.

| | | |
|---|---|---|
| BF2: | 5′-GCTTGYGACAAGGACTCYCA-3′ | (SEQ ID NO: 55) |
| BR1: | 5′-GTTYCTACTYCAGAGYGAT-3′ | (SEQ ID NO: 56) |

PCR reaction solution was prepared by admixing 0.4 μl of 50× Advantage 2 Polymerase Mix (CLONTECH), 2 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 1.6 μl of dNTP mixture (2.5 mM each, Takara Shuzo), 0.4 μl of 10 μM primers BF2 and BR1, 2 μl of template cDNA, and 13.2 μl of distilled water. The reaction condition was 95° C. for 1 minute for the starting denaturation, then a cycle set to include 95° C. for 30 seconds followed by 55° C. for 1 minute and 68° C. for 1 minute, which was repeated 40 times, and finally, extension reaction at 68° C. for 5 minutes.

The DNA fragment obtained was cloned in accordance with the method described in the attached manual using the TOPO TA Cloning Kit (Invitrogen). The cloned DNA sequence was decoded by using ABI377 DNA sequencer, thereby partial sequence represented by SEQ ID NO: 57 was obtained.

From the above-mentioned information on the sequence, primers RB5-1 (SEQ ID NO: 58) and RB5-3 (SEQ ID NO: 59) were prepared. Then the 5′ RACE experiment described below was carried out.

| | |
|---|---|
| RB5-1: | |
| 5′-GTGCATCCTCCGCCCCCAAAATGGAA-3′ | (SEQ ID NO: 58) |
| RB5-3: | |
| 5′-GACAGCGCAGCACATTCCTCCTCCACAC-3′ | (SEQ ID NO: 59) |

PCR reaction solution of 5′ RACE was prepared by admixing 1 μl of 50× Advantage 2 Polymerase Mix (CLONTECH), 5 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 4 μl of dNTP mixture (2.5 mM each, Takara Shuzo), 1 μl of 10 μM primer RB5-1, 1 μl of 10 μM primer AP1 (the primer AP1 is attached with Marathon Ready cDNA Kit by CLONTECH), 5 μl of template cDNA (CLONTECH, rat testis Marathon Ready cDNA), and 33 μl of distilled water. The reaction condition was 94° C. for 1 minute for the starting denaturation, then a cycle set to include 94° C. for 30 seconds followed by 72° C. for 3 minute, which was repeated 5 times, 94° C. for 30 seconds followed by 70° C. for 3 minute, which was repeated 5 times, and 94° C. for 30 seconds followed by 68° C. for 3 minute, which was repeated 25 times.

Next, using the PCR reaction solution described above as a template, nested PCR was performed. The reaction solution was-prepared by admixing 1 μl of 50× Advantage 2 Polymerase Mix (CLONTECH), 5 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 4 μl of dNTP mixture (2.5 mM each, Takara Shuzo), 1 μl of 10 μM primer RB5-3, 1 μl of 10 μM primer AP2 (the primer AP2 is attached with Marathon Ready cDNA Kit by CLONTECH), 5 μl of template cDNA (the PCR reaction solution diluted to 50 fold), and 33 μl of distilled water. The reaction condition was 94° C. for 1 minute for the starting denaturation, then a cycle set to include 94° C. for 30 seconds followed by 68° C. for 3 minute, which was repeated 35 times.

The DNA fragment obtained was cloned in accordance with the method described in the attached manual using the TOPO TA Cloning Kit (Invitrogen). The cloned DNA sequence was decoded by using ABI377 DNA sequencer, thereby the 5′ sequence (SEQ ID NO: 60) was obtained.

From the information of SEQ ID NO: 57, primers RB3-1 (SEQ ID NO: 61) and RB3-2 (SEQ ID NO: 62) were prepared. Then the 3′ RACE experiment described below was carried out.

| | |
|---|---|
| RB3-1: | |
| 5′-GAGACAGCTGCCACCCCCTGACTCGGAA-3′ | (SEQ ID NO: 61) |
| RB3-2: | |
| 5′-GGCGGAGGATGCACCACACTTGTCCCTG-3′ | (SEQ ID NO: 62) |

PCR reaction solution of 3′ RACE was prepared by admixing 1 μl of 50× Advantage 2 Polymerase Mix (CLONTECH), 5 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 4 μl of dNTP mixture (2.5 mM each, Takara Shuzo), 1 μl of 10 μM primer RB3-1, 1 μl of 10 μM primer AP1 (the primer AP1 is attached with Marathon Ready cDNA Kit by CLONTECH), 5 μl of template cDNA (CLONTECH, rat testis Marathon Ready cDNA), and 33 μl of distilled water. The reaction condition was 94° C. for 1 minute for the starting denaturation, then a cycle set to include 94° C. for 30 seconds followed by 72° C. for 3 minute, which was repeated 5 times, 94° C. for 30 seconds followed by 70° C. for 3 minute, which was repeated 5 times, 94° C. for 30 seconds followed by 68° C. for 3 minute, which was repeated 25 times.

Next, using the PCR reaction solution described above as a template, nested PCR was performed. The reaction solution was prepared by admixing 1 μl of 50× Advantage 2 Polymerase Mix (CLONTECH), 5 μl of attached 10× Advantage 2 PCR buffer (400 mM Tricine-KOH, 150 mM KOAc, 35 mM Mg(OAc)₂, 37.5 μg/ml BSA, 0.05% Tween-20, 0.05% Nonidet-P40), 4 μl of dNTP mixture (2.5 mM each, Takara Shuzo), 1 μl of 10 μM primer RB3-2, 1 μl of 10 μM primer AP2 (the primer AP2 is attached with Marathon Ready cDNA Kit by CLONTECH), 5 μl of template cDNA (the PCR reaction solution diluted to 50 fold), and 33 μl of distilled water. The reaction condition was 94° C. for 1 minute for the starting denaturation, then a cycle set to include 94° C. for 30 seconds followed by 68° C. for 3 minute, which was repeated 35 times.

The DNA fragment obtained was cloned in accordance with the method described in the attached manual using the TOPO TA Cloning Kit (Invitrogen). The cloned DNA sequence was decoded by using ABI377 DNA sequencer, thereby the 3′ sequence (SEQ ID NO: 63) was obtained.

From the information of 5′ RACE and 3′ RACE, primers RBv8-WF1 (SEQ ID NO: 64), RBv8-WF2 (SEQ ID NO: 65), RBv8-WR1 (SEQ ID NO: 66) and RBv8-WR2 (SEQ ID NO: 67) were prepared using rat brain Marathon-Ready cDNA (CLONTECH) as a template. Then the PCR reaction described below was carried out.

| | |
|---|---|
| RBv8-WF1: | |
| 5′-TAACCGCCACCGCCTCCT-3′ | (SEQ ID NO: 64) |
| RBv8-WF2: | |
| 5′-GGGACGCCATGGAGGAC-3′ | (SEQ ID NO: 65) |
| RBv8-WR1: | |
| 5′-CGAGACTTGACAGACATTGTTCAGTG-3′ | (SEQ ID NO: 66) |
| RBv8-WR2: | |
| 5′-TTTCCAGCTCCTGCTTCAGA-3′ | (SEQ ID NO: 67) |

The PCR reaction solution was prepared by admixing 0.6 μl of PfuTurbo DNA polymerase (Stratagene), 3 μl of attached 10× PCR buffer, 2.4 μl of 2.5 mM dNTP mixture, 1.5 μl each of 10 μM primers RBv8-WF1 and RBv8-WR1, 3 μl of template DNA and 18 μl of distilled water. The reaction condition was 95° C. for 1 minute for the starting denaturation, then a cycle set to include 95° C. for 30 seconds followed by 55° C. for 30 seconds and 72° C. for 1 minute, which was repeated 35 times, and finally, extension reaction at 72° C. for 5 minutes.

Next, using a solution, wherein the PCR reaction solution described above was 50-fold diluted with distilled water, as a template, nested PCR was performed. The PCR reaction solution was prepared by admixing 0.6 μl of PfuTurbo DNA polymerase (Stratagene), 3 μl of attached 10× PCR buffer, 2.4 μl of 2.5 mM dNTP mixture, 1.5 μl each of 10 μM primers RBv8-WF2 and RBv8-WR2, 3 μl of template DNA and 18 μl of distilled water. The reaction condition was 95° C. for 1 minute for the starting denaturation, then a cycle set to include 95° C. for 30 seconds followed by 55° C. for 30 seconds and 72° C. for 1 minute, which was repeated 35 times, and finally, extension reaction at 72° C. for 5 minutes.

The DNA fragment obtained was cloned in accordance with the method described in the attached manual using the Zero Blunt TOPO PCR Cloning Kit (Invitrogen). The cloned DNA sequence was decoded by using ABI377 DNA sequencer, and clarified to have a 356 bp base sequence represented by SEQ ID NO: 68. The plasmid having a DNA fragment having a base sequence represented by SEQ ID NO: 68 was designated pRBv. Escherichia coli was transformed by the plasmid pRBv, then the transformants obtained were designated E. coli TOP10/pRBv.

From a result of analyzing the base sequence of this DNA fragment, it was clarified that the DNA fragment represented by SEQ ID NO: 68 contains a DNA (SEQ ID NO: 70) encoding rat Bv8 precursor peptide (107 amino acid residues) represented by SEQ ID NO: 69.

In addition, the base sequences represented by SEQ ID NO: 70 has a typical signal sequence. Moreover, it became clear that the DNA having a base sequence represented by SEQ ID NO: 70 contains a 243 bp DNA (SEQ ID NO: 72) encoding rat Bv8 mature peptide (81 amino acid residues) represented by SEQ ID NO: 71.

### INDUSTRIAL APPLICABILITY

The protein of the present invention, its partial peptides, or salts thereof and the DNA encoding the protein or its partial peptide can be used for; 1) determination of ligands; 2) preparation of antibodies and antisera; 3) construction of recombinant receptor protein expression systems; 4) development of the receptor binding assay systems using the expression systems and screening of pharmaceutical candidate compounds; 5) effecting drug design based on comparison with structurally similar ligand receptors; 6) reagents for preparation of probes and PCR primers for gene diagnosis; 7) production of transgenic animals; and 8) pharmaceutical drugs for the gene prophylaxis and gene therapy.

1. A G protein-coupled receptor protein containing the same or substantially the same amino acid sequence as the amino acid sequence represented by SEQ ID NO: 4 or SEQ ID NO: 11, or a salt thereof.

2. A G protein-coupled receptor protein according to claim 1 which contains an amino acid sequence represented by SEQ ID NO: 4, or a salt thereof.

3. A G protein-coupled receptor protein according to claim 1 which contains an amino acid sequence represented by SEQ ID NO: 11, or a salt thereof.

4. A partial peptide of the protein according to claim 1, or a salt thereof.

5. A polynucleotide containing a polynucleotide encoding the protein according to claim 1.

6. A polynucleotide according to claim 5, which is DNA.

7. The DNA according to claim 6, which contains a base sequence represented by SEQ ID NO: 3 or SEQ ID NO: 10.

8. A recombinant vector containing the polynucleotide according to claim 5.

9. A transformant transformed with the recombinant vector according to claim 8.

10. A method of manufacturing the protein or its salt according to claim 1, which comprises culturing the transformant according to claim 9, producing and accumulating the protein according to claim 1.

11. An antibody to the protein according to claim 1, the partial peptide according to claim 4, or a salt thereof.

12. A method of determining a ligand to the protein or its salt according to claim 1, which comprises using the protein according to claim 1, the partial peptide according to claim 4, or a salt thereof.

13. A method of screening a compound or its salt that alters the binding property between a ligand and the protein or its salt according to claim 1, which comprises using the protein according to claim 1, the partial peptide according to claim 4, or a salt thereof.

14. A kit of screening a compound or its salt that alters the binding property between a ligand and the protein or its salt according to claim 1, which comprises using the protein according to claim 1, the partial peptide according to claim 4, or a salt thereof.

15. A compound or its salt that alters the binding property between a ligand and the protein or its salt according to claim 1, which is obtainable using the screening method according to claim 13 or the screening kit according to claim 14.

16. A pharmaceutical comprising a compound or its salt that alters the binding property between a ligand and the protein or its salt according to claim 1, which is obtainable using the screening method according to claim 13 or the screening kit according to claim 14.

17. The pharmaceutical according to claim 16, which is a prophylactic or therapeutic agent for alimentary diseases.

18. A DNA that hybridizes to the DNA according to claim 6 under a highly stringent condition.

19. A prophylactic or therapeutic method for alimentary diseases, which comprises administering an effective amount of compound that alters a binding property between ligand and the protein according to claim 1 or its salt obtainable by using the screening method according to claim 13 or the screening kit according to claim 14, to mammals.

20. Use of the compound that alters a binding property between ligand and the protein according to claim 1 or its salt obtainable by using the screening method according to claim 13 or the screening kit according to claim 14 for manufacturing a prophylactic or therapeutic agent for alimentary diseases.


SEQUENCE LISTING (72 sequences)

SEQ ID NO: 1 - DNA, 30 bp, Artificial Sequence
  primer
gtcgacatggagaccactgtggggaccctg

SEQ ID NO: 2 - DNA, 30 bp, Artificial Sequence
  primer
actagtttatttcagtcggatgcagtccac

SEQ ID NO: 3 - DNA, 1182 bp, Rat
atggagaccactgtggggaccctgggcgagaataccacaaacactttcaccgacttcttttctgcacgtgatggcagtggagccgaaacctcccccttgccattcactttcagctatggtgactatgacatgccctcggatgaagaggaggatgtgaccaactctcggactttctttgctgccaagattgtcattggcatggctttggtgggcatcatgctggtgtgtggcatcggcaacttcatcttcatcactgcgctggcccgctacaaaaagcttcgcaacctcaccaacctgcttatcgccaacctggccatttcggacttcctggtagccatcgtgtgctgcccctttgagatggactactatgtggtacgccagctctcctgggagcacggccatgtcctgtgcgcctccgtcaactacttgcgcaccgtctccctctacgtgtccactaacgccctactggccattgccattgacaggtatctggccattgtgcacccgctgagaccgcggatgaagtgtcaaacggctgcaggcctgatcttcctggtgtggtctgtgtccatcctcatcgccatcccagccgcctacttcaccactgagacggtgttggtcatcgtggaaagccaggagaagatcttctgcggccagatctggccggtggatcagcagttctactacaggtcctatttccttttggtcttcggcctcgagttcgtgggtcctgtaatcgccatgaccctgtgctatgccagggtgtcccgagagctctggttcaaggcggtgcccggcttccagacagagcagatccgccggaggctgcgctgtcgccgacggacggtactggggctcgtgtgcgtcctttccgcctatgtgctgtgctgggctcccttctatggcttcaccatcgtgcgtgacttcttcccctccgtgtttgtgaaagagaagcactacctcaccgccttttatgtggtggagtgcatcgccatgagcaacagtatgatcaatacgctgtgctttgtgactgtcaggaataacaccagtaagtacctcaagaggatcctgcggctccagtggagggcctctcctagcgggagcaaggccagcgctgacctcgacctcaggaccacggggattcctgccacggaggaggtggactgcatccgactgaaataa

SEQ ID NO: 4 - protein, 393 aa, Rat
METTVGTLGENTTNTFTDFFSARDGSGAETSPLPFTFSYGDYDMPSDEEEDVTNSRTFFAAKIVIGMALVGIMLVCGIGNFIFITALARYKKLRNLTNLLIANLAISDFLVAIVCCPFEMDYYVVRQLSWEHGHVLCASVNYLRTVSLYVSTNALLAIAIDRYLAIVHPLRPRMKCQTAAGLIFLVWSVSILIAIPAAYFTTETVLVIVESQEKIFCGQIWPVDQQFYYRSYFLLVFGLEFVGPVIAMTLCYARVSRELWFKAVPGFQTEQIRRRLRCRRRTVLGLVCVLSAYVLCWAPFYGFTIVRDFFPSVFVKEKHYLTAFYVVECIAMSNSMINTLCFVTVRNNTSKYLKRILRLQWRASPSGSKASADLDLRTTGIPATEEVDCIRLK

SEQ ID NO: 5 - DNA, 31 bp, Artificial Sequence
  primer
cctcaccaayctgctyatygccaacctggcc

SEQ ID NO: 6 - DNA, 26 bp, Artificial Sequence
  primer
gtggtrcgscagctctcctgggagca

SEQ ID NO: 7 - DNA, 23 bp, Artificial Sequence
  primer
tcccgggagctctggttcaaggc

SEQ ID NO: 8 - DNA, 27 bp, Artificial Sequence
  primer
gagtgcatcgccatgagcaacagcatg

SEQ ID NO: 9 - DNA, 23 bp, Artificial Sequence
  primer
ggcttgaaccagagctcccggga

SEQ ID NO: 10 - DNA, 1266 bp, Rat
atggtatcagttctgtccaacagggacctccacacactggccccagctgaagtgctgaactccacgtgggcctatctccctgacacataccagcctacctgccacatcatcaacatgggagaccagaacggaaacacaagctttgcaccagacttgaacccaccccaagaccacgtctccttgctccccttaaactacagttatggagattatgacatccccctggatgacgatgaggatgtgaccaagacacagaccttctttgcagccaaaatcgtcattggcgtagccctggcaggcatcatgctagtctgcggcgttggcaactttgtcttcattgctgccctcgcccgctacaagaagctgcgcaaccttaccaacctcctcatcgctaacctggccatctctgacttcctggtggcgatcgtctgctgcccctttgagatggactactacgtagtacgtcagctttcctgggagcatggtcacgtgctttgtgcctccgtcaactaccttcgtacagtctccctgtacgtctccaccaatgctctgctggccatcgctattgacagatatctcgctattgtccaccccttaaaacggatgaattaccagaccgcctccttcctgatcgctttggtctggatggtctccatcctcatcgccatcccatctgcctacttcaccacagaaaccatccttgttatcgtcaagaatcaggaaaagctcttctgtggtcagatctggcccgtggaccagcagctctactacaaatcctacttcctcttcgtcttcgggcttgagttcgtgggtcccgtggtcactatgaccctgtgctatgccaggatctcccaggagctctggttcaaggctgtacctggtttccagacggagcagatccgcaagcgactgcgctgccgccgaaagacagtgctattgctcatgggtatcctcacagcctacgtgctgtgctgggcgcctttctatggctttaccatagtgcgagacttcttccccacgctggttgtgaaggagaagcactacctcaccgccttctatgtcgtcgagtgcatcgccatgagcaacagcatgatcaatactatatgcttcgtgacggtcaagaacaacaccatgaaatacttcaagaagatgctgctgctgcactggcggccctctcactacgggagtaagtccagcgcggacctcgacctcaaaaccagtggggttcctgccaccgaagaggtggactgtatcaggctaaagtag

SEQ ID NO: 11 - protein, 421 aa, Rat
MVSVLSNRDLHTLAPAEVLNSTWAYLPDTYQPTCHIINMGDQNGNTSFAPDLNPPQDHVSLLPLNYSYGDYDIPLDDDEDVTKTQTFFAAKIVIGVALAGIMLVCGVGNFVFIAALARYKKLRNLTNLLIANLAISDFLVAIVCCPFEMDYYVVRQLSWEHGHVLCASVNYLRTVSLYVSTNALLAIAIDRYLAIVHPLKRMNYQTASFLIALVWMVSILIAIPSAYFTTETILVIVKNQEKLFCGQIWPVDQQLYYKSYFLFVFGLEFVGPVVTMTLCYARISQELWFKAVPGFQTEQIRKRLRCRRKTVLLLMGILTAYVLCWAPFYGFTIVRDFFPTLVVKEKHYLTAFYVVECIAMSNSMINTICFVTVKNNTMKYFKKMLLLHWRPSHYGSKSSADLDLKTSGVPATEEVDCIRLK

SEQ ID NO: 12 - protein, 393 aa, Human
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 13 - DNA, 19 bp, Artificial Sequence
  primer
atgctggtgtgtggcatcg

SEQ ID NO: 14 - DNA, 20 bp, Artificial Sequence
  primer
ttgcgaagctttttgtagcg

SEQ ID NO: 15 - DNA, 27 bp, Artificial Sequence
  primer
caacttcatcttcatcactgcgctggc

SEQ ID NO: 16 - DNA, 21 bp, Artificial Sequence
  primer
cacagaccttctttgcagcca

SEQ ID NO: 17 - DNA, 17 bp, Artificial Sequence
  primer
cagactagcatgatgcc

SEQ ID NO: 18 - DNA, 24 bp, Artificial Sequence
  primer
aatcgtcattggcgtagccctggc

SEQ ID NO: 19 - DNA, 30 bp, Artificial Sequence; identical to SEQ ID NO: 1
  primer

SEQ ID NO: 20 - DNA, 30 bp, Artificial Sequence; identical to SEQ ID NO: 2
  primer

SEQ ID NO: 21 - DNA, 29 bp, Artificial Sequence
  primer
gtcgacatggtatcagttctgtccaacag

SEQ ID NO: 22 - DNA, 30 bp, Artificial Sequence
  primer
actagtctactttagcctgatacagtccac

SEQ ID NO: 23 - DNA, 23 bp, Artificial Sequence
  primer
tcaccycaagtgaycatgagagg

SEQ ID NO: 24 - DNA, 25 bp, Artificial Sequence
  primer
ctaaaarttgryrttcttcaagtcc

SEQ ID NO: 25 - DNA, 20 bp, Artificial Sequence
  primer
atcacaggggcctgtgarcg

SEQ ID NO: 26 - DNA, 20 bp, Artificial Sequence
  primer
agcagcggtacctgccgtcc

SEQ ID NO: 27 - DNA, 186 bp, Rat
agatgtccagtgtggggctggcacctgctgtgctatcagcctgtggctgcggggcctgaggctgtgtacccctctggggcgggaaggagaggagtgccaccctggaagccacaagatccctttctttaggaaacgccaacaccatacctgtccctgttcacccagcctgctgtgctccaggttccc

SEQ ID NO: 28 - DNA, 186 bp, Rat
agatgtccagtgtggggctggcacctgctgtgctatcagcctgtggctgcggggcctgaggctgtgcacccctctggggcgggaaggagaggagtgccaccctggaagccacaagatccctttctttaggaaacgccaacaccatacctgtccctgttcacccagcctgctgtgctccaggttccc

SEQ ID NO: 29 - DNA, 28 bp, Artificial Sequence
  primer
gtggcactcctctccttcccgccccaga

SEQ ID NO: 30 - DNA, 28 bp, Artificial Sequence
  primer
caggccccgcagccacaggctgatagca

SEQ ID NO: 31 - DNA, 28 bp, Artificial Sequence
  primer
agcaggtgccagccccacactggacatc

SEQ ID NO: 32 - DNA, 244 bp, Rat
agagagatgaggcatttagaggcagccctggatccgactatataaatctgaaggaggtaaggtaggacagcttggccttcttagcttgtctagtgcaaggcagtgcagaaggaagtgagggattccagagtggacagtgtttgccttcaccccaagtgatcatgagaggtgctgtgcaagtcttcatcatgctccttctagcaactgtctctgactgtgcggtgatcacaggggcctgtgaacg

SEQ ID NO: 33 - DNA, 27 bp, Artificial Sequence
  primer
ggaaggagaggagtgccaccctggaag

SEQ ID NO: 34 - DNA, 28 bp, Artificial Sequence
  primer
accatacctgtccctgttcacccagcct

SEQ ID NO: 35 - DNA, 464 bp, Rat
ctgttcacccagcctgctgtgctccaggttcccagatggcaggtaccgctgctcccaggacttgaagaatgtcaacttttagtttatctggactctgtctgggtccctactgggtgacctcttgtgttacatctgtgtgacttagttccgtgcaacttctccactccccaccctgtccgtgtgtgtgcagacaagcatatcttccactacggaacagtccagcagcgtgcagagaggagtttgcagccttgagaagtgggccagcctggccttcctggccagaccgcctgaagttgtgacactgggacctcctcaattgtctgcccttcctgcatgtgcccttctccctaaaccacacctcccaggccctggcctgtgggtgcgtcactaagtcacggggtctatggggggaagatcaacattctcctcattttctttcattggctagctccttgttttaggag

SEQ ID NO: 36 - DNA, 30 bp, Artificial Sequence
  primer
attccagagtggacagtgtttgccttcacc

SEQ ID NO: 37 - DNA, 28 bp, Artificial Sequence
  primer
gatcatgagaggtgctgtgcaagtcttc

SEQ ID NO: 38 - DNA, 26 bp, Artificial Sequence
  primer
ctctctgcacgctgctggactgttcc

SEQ ID NO: 39 - DNA, 30 bp, Artificial Sequence
  primer
cagatgtaacacaagaggtcacccagtagg

SEQ ID NO: 40 - DNA, 375 bp, Rat
gatcatgagaggtgctgtgcaagtcttcatcatgctccttctagcaactgtctctgactgtgcggtgatcacaggggcctgtgaacgagatgtccagtgtggggctggcacctgctgtgctatcagcctgtggctgcggggcctgaggctgtgtacccctctggggcgggaaggagaggagtgccaccctggaagccacaagatccctttctttaggaaacgccaacaccatacctgtccctgttcacccagcctgctgtgctccaggttcccagatggcaggtaccgctgctcccaggacttgaagaatgtcaacttttagtttatctggactctgtctgggtccctactgggtgacctcttgtgttacatctg

SEQ ID NO: 41 - DNA, 375 bp, Rat
gatcatgagaggtgctgtgcaagtcttcatcatgctccttctagcaactgtctctgactgtgcggtgatcacaggggcctgtgaacgagatgtccagtgtggggctggcacctgctgtgctatcagcctgtggctgcggggcctgaggctgtgtacccctctggggcgggaaggagaggagtgccaccctggaagctacaagatccctttctttaggaaacgccaacaccatacctgtccctgttcacccagcctgctgtgctccaggttcccagatggcaggtaccgctgctcccaggacttgaagaatgtcaacttttagtttatctggactctgtctgggtccctactgggtgacctcttgtgttacatctg

SEQ ID NO: 42 - DNA, 375 bp, Rat
gatcatgagaggtgctgtgcaagtcttcatcatgctccttctagcaactgtctctgactgtgcggtgatcacaggggcctgtgaacgagatgtccagtgtggggctggcacctgctgtgctatcagcctgtggctgcggggcctgaggctgtgtacccctctggggcaggaaggagaggagtgccaccctggaagccacaagatccctttctttaggaaacgccaacaccatacctgtccctgttcacccagcctgctgtgctccaggttcccagatggcaggtaccgctgctcccaggacttgaagaatgtcaacttttagtttatctggactctgtctgggtccctactgggtgacctcttgtgttacatctg

SEQ ID NO: 43 - protein, 105 aa, Rat
MRGAVQVFIMLLLATVSDCAVITGACERDVQCGAGTCCAISLWLRGLRLCTPLGREGEECHPGSHKIPFFRKRQHHTCPCSPSLLCSRFPDGRYRCSQDLKNVNF

SEQ ID NO: 44 - DNA, 315 bp, Rat
atgagaggtgctgtgcaagtcttcatcatgctccttctagcaactgtctctgactgtgcggtgatcacaggggcctgtgaacgagatgtccagtgtggggctggcacctgctgtgctatcagcctgtggctgcggggcctgaggctgtgtacccctctggggcgggaaggagaggagtgccaccctggaagccacaagatccctttctttaggaaacgccaacaccatacctgtccctgttcacccagcctgctgtgctccaggttcccagatggcaggtaccgctgctcccaggacttgaagaatgtcaacttt

SEQ ID NO: 45 - protein, 105 aa, Rat
MRGAVQVFIMLLLATVSDCAVITGACERDVQCGAGTCCAISLWLRGLRLCTPLGREGEECHPGSYKIPFFRKRQHHTCPCSPSLLCSRFPDGRYRCSQDLKNVNF

SEQ ID NO: 46 - DNA, 315 bp, Rat
atgagaggtgctgtgcaagtcttcatcatgctccttctagcaactgtctctgactgtgcggtgatcacaggggcctgtgaacgagatgtccagtgtggggctggcacctgctgtgctatcagcctgtggctgcggggcctgaggctgtgtacccctctggggcgggaaggagaggagtgccaccctggaagctacaagatccctttctttaggaaacgccaacaccatacctgtccctgttcacccagcctgctgtgctccaggttcccagatggcaggtaccgctgctcccaggacttgaagaatgtcaacttt

SEQ ID NO: 47 - protein, 105 aa, Rat
MRGAVQVFIMLLLATVSDCAVITGACERDVQCGAGTCCAISLWLRGLRLCTPLGQEGEECHPGSHKIPFFRKRQHHTCPCSPSLLCSRFPDGRYRCSQDLKNVNF

SEQ ID NO: 48 - DNA, 315 bp, Rat
atgagaggtgctgtgcaagtcttcatcatgctccttctagcaactgtctctgactgtgcggtgatcacaggggcctgtgaacgagatgtccagtgtggggctggcacctgctgtgctatcagcctgtggctgcggggcctgaggctgtgtacccctctggggcaggaaggagaggagtgccaccctggaagccacaagatccctttctttaggaaacgccaacaccatacctgtccctgttcacccagcctgctgtgctccaggttcccagatggcaggtaccgctgctcccaggacttgaagaatgtcaacttt

SEQ ID NO: 49 - protein, 86 aa, Rat
AVITGACERDVQCGAGTCCAISLWLRGLRLCTPLGREGEECHPGSHKIPFFRKRQHHTCPCSPSLLCSRFPDGRYRCSQDLKNVNF

SEQ ID NO: 50 - DNA, 258 bp, Rat
gcggtgatcacaggggcctgtgaacgagatgtccagtgtggggctggcacctgctgtgctatcagcctgtggctgcggggcctgaggctgtgtacccctctggggcgggaaggagaggagtgccaccctggaagccacaagatccctttctttaggaaacgccaacaccatacctgtccctgttcacccagcctgctgtgctccaggttcccagatggcaggtaccgctgctcccaggacttgaagaatgtcaacttt

SEQ ID NO: 51 - protein, 86 aa, Rat
AVITGACERDVQCGAGTCCAISLWLRGLRLCTPLGREGEECHPGSYKIPFFRKRQHHTCPCSPSLLCSRFPDGRYRCSQDLKNVNF

SEQ ID NO: 52 - DNA, 258 bp, Rat; identical to SEQ ID NO: 50

SEQ ID NO: 53 - protein, 86 aa, Rat
AVITGACERDVQCGAGTCCAISLWLRGLRLCTPLGQEGEECHPGSHKIPFFRKRQHHTCPCSPSLLCSRFPDGRYRCSQDLKNVNF

SEQ ID NO: 54 - DNA, 258 bp, Rat
gcggtgatcacaggggcctgtgaacgagatgtccagtgtggggctggcacctgctgtgctatcagcctgtggctgcggggcctgaggctgtgtacccctctggggcaggaaggagaggagtgccaccctggaagccacaagatccctttctttaggaaacgccaacaccatacctgtccctgttcacccagcctgctgtgctccaggttcccagatggcaggtaccgctgctcccaggacttgaagaatgtcaacttt

SEQ ID NO: 55 - DNA, 20 bp, Artificial Sequence
  primer
gcttgygacaaggactcyca

SEQ ID NO: 56 - DNA, 19 bp, Artificial Sequence
  primer
gttyctactycagagygat

SEQ ID NO: 57 - DNA, 210 bp, Rat
gtgtggaggaggaatgtgctgcgctgtcagtatctgggttaagagcataaggatctgcacacctatgggccaagtgggagacagctgccaccccctgactcggaaagttccattttgggggcggaggatgcaccacacttgtccctgcctgccaggtttggcatgtttaaggacttctttcaaccgttttatttgtttggcccggaagtg

SEQ ID NO: 58 - DNA, 26 bp, Artificial Sequence
  primer
gtgcatcctccgcccccaaaatggaa

SEQ ID NO: 59 - DNA, 28 bp, Artificial Sequence
  primer
gacagcgcagcacattcctcctccacac

SEQ ID NO: 60 - DNA, 148 bp, Human
cgcgtccctaaccgccaccgcctcctcgggacgccatggaggacccgcgctgtgccccgctactgctacttttgctgctaccgctgctgctcacaccgcccgccggggatgccgcggtcatcaccggggcttgcgacaaggactctca

SEQ ID NO: 61 - DNA, 28 bp, Artificial Sequence
  primer
gagacagctgccaccccctgactcggaa

SEQ ID NO: 62 - DNA, 28 bp, Artificial Sequence
  primer
ggcggaggatgcaccacacttgtccctg

SEQ ID NO: 63 - DNA, 150 bp, Rat
cctgcctgccaggtttggcatgtttaaggacttctttcaaccgttttatttgtttggcccggaagtgatcactctgaagcaggagctggaaatgtgaacctctactcactgaacaatgtctgtcaagtctcgcttgtaattgtgtcaaag

SEQ ID NO: 64 - DNA, 18 bp, Artificial Sequence
  primer
taaccgccaccgcctcct

SEQ ID NO: 65 - DNA, 17 bp, Artificial Sequence
  primer
gggacgccatggaggac

SEQ ID NO: 66 - DNA, 26 bp, Artificial Sequence
  primer
cgagacttgacagacattgttcagtg

SEQ ID NO: 67 - DNA, 20 bp, Artificial Sequence
  primer
tttccagctcctgcttcaga

SEQ ID NO: 68 - DNA, 356 bp, Rat
gggacgccatggaggacccgcgctgtgccccgctactgctacttttgctgctaccgctgctgctcacaccgcccgccggggatgccgcggtcatcaccggggcttgcgacaaggactctcagtgtggaggaggaatgtgctgcgctgtcagtatctgggttaagagcataaggatctgcacacctatgggccaagtgggagacagctgccaccccctgactcggaaagttccattttgggggcggaggatgcaccacacttgtccctgcctgccaggtttggcatgtttaaggacttctttcaaccgttttatttgtttggcccggaagtgatcactctgaagcaggagctggaaa

SEQ ID NO: 69 - protein, 107 aa, Rat
MEDPRCAPLLLLLLLPLLLTPPAGDAAVITGACDKDSQCGGGMCCAVSIWVKSIRICTPMGQVGDSCHPLTRKVPFWGRRMHHTCPCLPGLACLRTSFNRFICLARK

SEQ ID NO: 70 - DNA, 321 bp, Rat
atggaggacccgcgctgtgccccgctactgctacttttgctgctaccgctgctgctcacaccgcccgccggggatgccgcggtcatcaccggggcttgcgacaaggactctcagtgtggaggaggaatgtgctgcgctgtcagtatctgggttaagagcataaggatctgcacacctatgggccaagtgggagacagctgccaccccctgactcggaaagttccattttgggggcggaggatgcaccacacttgtccctgcctgccaggtttggcatgtttaaggacttctttcaaccgttttatttgtttggcccggaag

SEQ ID NO: 71 - protein, 81 aa, Rat
AVITGACDKDSQCGGGMCCAVSIWVKSIRICTPMGQVGDSCHPLTRKVPFWGRRMHHTCPCLPGLACLRTSFNRFICLARK

SEQ ID NO: 72 - DNA, 243 bp, Rat
gcggtcatcaccggggcttgcgacaaggactctcagtgtggaggaggaatgtgctgcgctgtcagtatctgggttaagagcataaggatctgcacacctatgggccaagtgggagacagctgccaccccctgactcggaaagttccattttgggggcggaggatgcaccacacttgtccctgcctgccaggtttggcatgtttaaggacttctttcaaccgttttatttgtttggcccggaag